(19) United States
(12) Patent Application Publication
(10) Pub. No.: US 2025/0263400 A1
(43) Pub. Date: Aug. 21, 2025

(54) GCN2 MODULATOR COMPOUNDS

(71) Applicant: Alesta Therapeutics BV, Leiden (NL)

(72) Inventor: Joseph VACCA, Telford, PA (US)

(21) Appl. No.: 18/854,879

(22) PCT Filed: Apr. 7, 2023
(86) PCT No.: PCT/US2023/017978

§ 371 Date: Oct. 7, 2024

Related U.S. Application Data
(60) Provisional application No. 63/329,281, filed on Apr. 8, 2022.
(60) Provisional application No. 63/329,282, filed on Apr. 8, 2022.

(51) Int. Cl. C07D 403/12; A61K 31/517; C07D 239/84; C07D 401/04; C07D 401/12; C07D 401/14; C07D 409/12; C07D 409/14

(52) U.S. Cl. CPC C07D 403/12; A61K 31/517; C07D 239/84; C07D 401/04; C07D 401/12; C07D 401/14; C07D 409/12; C07D 409/14

(57) ABSTRACT

The disclosures herein relate to compounds of Formula (I) and Formula (Ia). or a stereoisomer, tautomer, or a pharmaceutically acceptable salt thereof, wherein variables are defined herein, and their use in treating, preventing, ameliorating, controlling or reducing the risk of disorders associated with General Control Nondepressible 2 (GCN2).

### CROSS REFERENCE TO RELATED APPLICATIONS

This application claims priority to U.S. Provisional Application Ser. No. 63/329,281, filed Apr. 8, 2022, and U.S. Provisional Application Ser. No. 63/329,282, filed Apr. 8, 2022, both of which are hereby incorporated by reference in its entirety.

### FIELD OF THE INVENTION

This application relates to novel compounds and their use as General Control Nondepressible 2 (GCN2) modulators. Compounds described herein may be useful in the treatment or prevention of diseases in which GCN2 is involved.

### BACKGROUND

Diverse cellular conditions and stresses activate a widely conserved signalling pathway termed the Integrated Stress Response (ISR) pathway. The activation of ISR can trigger cell-cycle arrest, differentiation, amino acid biosynthetic and transport pathways, compensatory adaptation, or apoptosis, depending on the cell type and the initiating stress. General Control.

Nonderepressible 2 (GCN2) is one of four stress kinases of the ISR pathway that senses amino acid availability and controls gene expression in response to amino acid deprivation, UV-irradiation, viral infection, proteasome inhibition, hypoxia, glucose deprivation and oxidative stress. In mammals, GCN2 is also called EIF2AK4 (eukaryotic translation initiation factor 2 alpha kinase 4).

GCN2 harbours a eukaryotic kinase domain, a pseudo-kinase domain and a histidyl-tRNA synthetase (HisRS)-related domain, which binds uncharged tRNAs with higher affinity than it does charged tRNAs. Sequences at both the N- and C-termini of GCN2 have been shown to be important for efficient sensing of the starvation signal. Lysine residues in the C-terminus have also been shown to be required for tRNA binding and kinase activity and residues at the tip of the C-terminal region confer ribosome binding capabilities on GCN2, which are important for translational control.

In eukaryotes, the mechanism for recognizing indispensable amino acid deficiency follows the conserved general control system, wherein uncharged transfer RNA induces first the autophosphorylation of GCN2 and then the phosphorylation of eukaryotic initiation factor 2α (eIF2α), leading to reduced gdlobal protein synthesis and thus to reduced overall utilization of amino acids. Simultaneously, a group of stress-responsive mRNAs with upstream open reading frames (uORF) that include ATF4, CHOP, GADD34 and the (β-secretase BACE-1, are translated more efficiently when eIF2α is phosphorylated, which in turn increases amino acid biosynthetic and transport pathways. GCN2-mediated translational program also controls host responses to infection, responses to immunization, inflammation and other physiological and pathological processes. A significant subset of the genes upregulated by ATF4 are involved in amino acid import and metabolism and a hallmark of ATF4−/−cells is their impaired metabolism of amino acids.

Therefore, modulation of general control nonderepressible 2 (GCN2) may provide a therapeutic strategy for many diseases.

### SUMMARY OF THE INVENTION

The present invention provides compounds having activity as general control nonderepressible 2 (GCN2) modulators.

In one aspect, the invention provides a compound of Formula (I):

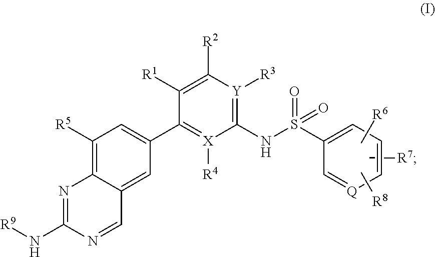

or a stereoisomer, tautomer, or a pharmaceutically acceptable salt thereof, wherein:
R¹, R², R³ and R⁴ are independently selected from the group consisting of H, halo and C₁₋₃ alkyl;
R⁵ is H or C₁₋₃ alkyl;
R⁶, R⁷ and R⁸ are independently selected from the group consisting of H, halo, C₁. 6 alkyl, and C₁₋₆ alkoxy, wherein the C₁₋₆ alkyl is optionally substituted with OH and the C₁₋₆ alkoxy is optionally substituted with C₁₋₆ alkoxy or NR¹⁰R¹¹;
R⁹ is H, C₁₋₆ alkyl, C₅₋₆ cycloalkyl or 4- to 6-membered heterocyclyl, wherein the C₁₋₆ alkyl is substituted with NR¹⁰R¹¹, and the C₅₋₆ cycloalkyl and 4- to 6-membered heterocyclyl are optionally substituted with one or two substituents each independently selected from the group consisting of oxo, NR¹⁰R¹¹, —C(O)N¹⁰R¹¹, and C₁₋₆ alkyl optionally substituted with NR¹⁰R¹¹;
R¹⁰ and R¹¹ are independently H or C₁₋₃ alkyl optionally substituted with C₁₋₆ alkoxy; and
X, Y and Q are independently C, CH or N,
provided that when R⁹ is H, and X and Y are C or CH, then at least one of R⁶, R⁷ and R⁸ is C₁₋₆ alkoxy, wherein the C₁₋₆ alkoxy is substituted with C₁₋₆ alkoxy or NR¹⁰R¹¹.

In some embodiments, R⁹ is H, C₁₋₆ alkyl, C₅₋₆ cycloalkyl or 5- or 6-membered heterocyclyl, wherein the C₁₋₆ alkyl is substituted with NR¹⁰R¹¹, and the C₅₋₆ cycloalkyl and 5 or 6-membered heterocyclyl are optionally substituted with one or two substituents each independently selected from the group consisting of oxo, NR¹⁰R¹¹, —C(O)NR¹⁰R¹¹, and C₁₋₆ alkyl optionally substituted with NR¹⁰R¹¹.

In another aspect, the invention provides a compound of Formula (Ia):

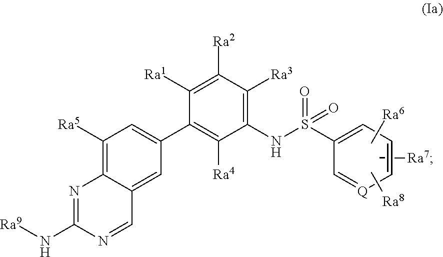

or a stereoisomer, tautomer, or a pharmaceutically acceptable salt thereof, wherein:
Ra¹ and Ra⁴ are halo;
Ra² and Ra³ are independently selected from the group consisting of H, halo and C₁₋₃ alkyl;
Ra⁵ is H or C₁₋₃ alkyl;
Ra⁶, R⁷ and R⁸ are independently selected from the group consisting of H, halo, C₁₋₆ alkyl, and C₁₋₆ alkoxy, wherein the C₁₋₆ alkyl and C₁₋₆ alkoxy are optionally substituted with OH;
Ra⁹ is H, C₅₋₆ cycloalkyl or 5 or 6-membered heterocyclyl, wherein the C₅₋₆ cycloalkyl and 5 or 6-membered heterocyclyl are optionally substituted with OH; and
Q is C, CH or N.

Compounds of the present invention may be used as GCN2 modulators. Compounds of the present invention may be used as GCN2 inhibitors. Compounds of the present invention may be used as GCN2 antagonists. Compounds of the present invention may be used as GCN2 agonists. Compounds of the present invention may be used in the treatment of a disease or disorder characterised by activation of GCN2. Compounds of the present invention may be used in the manufacture of medicaments. The compounds or medicaments may be for use in treating, preventing, ameliorating, controlling or reducing the risk of diseases or disorders in which GCN2 is involved. Compounds of the present invention may be for use as a single agent or in combination with one or more additional pharmaceutical agents. Additional pharmaceutical agents may include radiotherapy, chemotherapy, immunotherapy or tumor microenvironment modulating agents. Compounds of the present invention may be useful in the treatment of cancer, neurodegenerative diseases, chronic infections or conditions or symptoms related thereto. Compounds of the present invention may be useful in the treatment of breast cancer, colorectal cancer, ovarian cancer, prostate cancer, pancreatic cancer, kidney cancer, lung cancer, melanoma, fibrosarcoma, bone sarcoma, connective tissue sarcoma, renal cell carcinoma, giant cell carcinoma, squamous cell carcinoma, leukemia, skin cancer, soft tissue cancer, liver cancer, gastrointestinal carcinoma, adenocarcinoma, hepatocellular carcinoma, thyroid cancer, multiple myeloma, cancer of secretory cells, myelodysplastic syndrome, myeloproliferative neoplasm, malignant glioma, non-Hodgkin's lymphoma, Hodgkin's lymphoma, Burkitt's lymphoma, chronic lymphocytic leukemia, chronic myeloid leukemia, hairy cell leukemia, monoclonal gammopathy of undetermined significance (MGUS), plasmacytoma, lymphoplasmacytic lymphoma, acute lymphoblastic leukemia, acute myeloid leukemia, chronic myelomonocytic leukemia, juvenile myelomonocytic leukemia, large granular lymphocytic leukemia, B-cell prolymphocytic leukemia, T-cell prolymphocytic leukemia. small cell lung cancer, malignant pleural mesothelioma, Head and neck squamous cell carcinoma, glioblastoma multiforme, sarcoma, pediatric neuroblastoma or symptoms related thereto.

### DETAILED DESCRIPTION OF THE INVENTION

The invention relates to novel compounds. The invention also relates to the use of novel compounds as modulators of GCN2. The invention further relates to the use of novel compounds in the manufacture of medicaments for use as GCN2 modulators.

The invention further relates to compounds, compositions and medicaments that may be useful in the treatment of cancer, neurodegenerative diseases, chronic infections or conditions or symptoms related thereto.

In one aspect, provided is a compound of Formula (I):

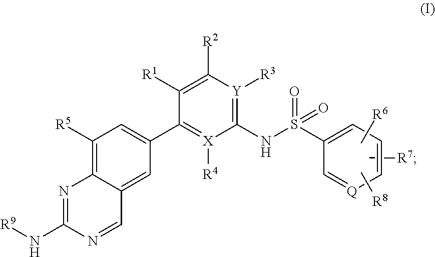

or a stereoisomer, tautomer, or a pharmaceutically acceptable salt thereof, wherein:
R¹, R², R³ and R⁴ are independently selected from the group consisting of H, halo and C₁₋₃ alkyl;
R⁵ is H or C₁₋₃ alkyl;
R⁶, R⁷ and R⁸ are independently selected from the group consisting of H, halo, C₁₋₆ alkyl, and C₁₋₆ alkoxy, wherein the C₁₋₆ alkyl is optionally substituted with OH and the C₁₋₆ alkoxy is optionally substituted with C₁₋₆ alkoxy or NR¹⁰R¹¹;
R⁹ is H, C₁₋₆ alkyl, C₅₋₆ cycloalkyl or 4- to 6-membered heterocyclyl, wherein the C₁₋₆ alkyl is substituted with NR¹⁰R¹¹, and the C₅₋₆ cycloalkyl and 4- to 6-membered heterocyclyl are optionally substituted with one or two substituents each independently selected from the group consisting of oxo, NR¹⁰R¹¹, —C(O)NR¹⁰R¹¹, and C₁₋₆ alkyl optionally substituted with NR¹⁰R¹¹;
R¹⁰ and R¹¹ are independently H or C₁₋₃ alkyl optionally substituted with C₁₋₆ alkoxy; and
X, Y and Q are independently C, CH or N,
provided that when R⁹ is H, and X and Y are C or CH, then at least one of R⁶, R⁷ and R⁸ is C₁₋₆ alkoxy, wherein the C₁₋₆ alkoxy is substituted with C₁₋₆ alkoxy or NR¹⁰R¹¹.

In the descriptions herein, it is understood that every description, variation, embodiment, or aspect of a moiety may be combined with every description, variation, embodiment, or aspect of other moieties the same as if each and every combination of descriptions is specifically and individually listed.

In some embodiments, R² and R³ are H.

In some embodiments, at least one of R⁶, R⁷ and R⁸ is not H. In some embodiments, one of R⁶, R⁷ and R⁸ is selected from the group consisting of halo, C₁₋₆ alkyl, and C₁₋₆ alkoxy, wherein the C₁₋₆ alkyl is optionally substituted with OH and the C₁₋₆ alkoxy is optionally substituted with C₁₋₆ alkoxy or NR¹⁰R¹¹, and the remainder of R⁶, R⁷ and R⁸ are H. In some embodiments, two of R⁶, R⁷ and R⁸ are independently selected from the group consisting of halo, C₁₋₆ alkyl, and C₁₋₆ alkoxy, wherein the C₁₋₆ alkyl is optionally substituted with OH and the C₁₋₆ alkoxy is optionally substituted with C₁₋₆ alkoxy or NR¹⁰R¹¹, and the remainder is H. In some embodiments, R⁶, R⁷ and R⁸ are each independently selected from the group consisting of halo, C₁₋₆ alkyl, and C₁₋₆ alkoxy, wherein the C₁₋₆ alkyl is optionally substituted with OH and the C₁₋₆ alkoxy is optionally substituted with C₁₋₆ alkoxy or NR¹⁰R¹¹.

In some embodiments, the moiety:
is selected from the group consisting of:

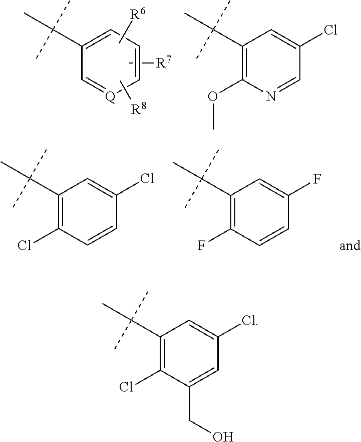

In some embodimentns, the moiety is

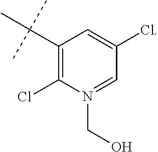

In some embodiments, R⁹ is H. In some embodiments, R⁹ is C₁₋₆ alkyl substituted with NR¹⁰R¹¹. In some embodiments, R⁹ is C₅₋₆ cycloalkyl or 4- to 6-membered heterocyclyl, wherein C₅₋₆ cycloalkyl and 4- to 6-membered heterocyclyl are optionally substituted with one or two substituents each independently selected from the group consisting of oxo, NR¹⁰R¹¹, —C(O)NR¹⁰R¹¹, and C₁₋₆ alkyl optionally substituted with NR¹⁰R¹¹. In some embodiments, R⁹ is

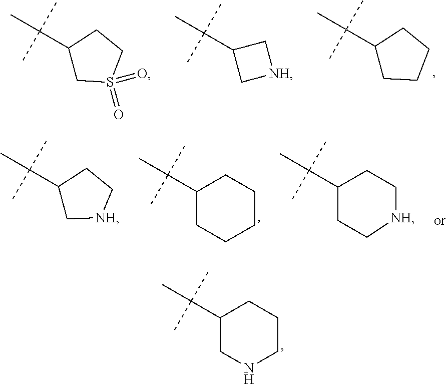

In some embodiments, R⁹ is

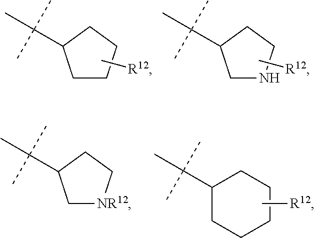

wherein R¹² is selected from the group consisting NR¹⁰R¹¹, —C(O)NR¹⁰R¹¹, or C₁₋₆ alkyl optionally substituted with NR¹⁰R¹¹.

In some embodiments, X, Y or both are N.

In some embodiments, provided is a compound of Formula (II):

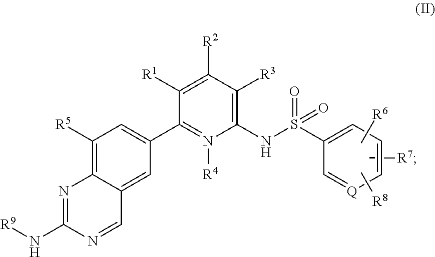

or a stereoisomer, tautomer, or a pharmaceutically acceptable salt thereof, wherein:
R¹, R², R³ and R⁴ are independently selected from the group consisting of H, halo and C₁₋₃ alkyl;
R⁵ is H or C₁₋₃ alkyl;
R⁶, R⁷ and R⁸ are independently selected from the group consisting of H, halo, C₁₋₆ alkyl, and C₁₋₆ alkoxy, wherein the C₁₋₆ alkyl is optionally substituted with OH and the C₁₋₆ alkoxy is optionally substituted with C₁₋₆ alkoxy or NR¹⁰R¹¹;
R⁹ is H, C₁₋₆ alkyl, C₅₋₆ cycloalkyl or 4- to 6-membered heterocyclyl, wherein the C₁₋₆ alkyl is substituted with NR¹⁰R¹¹, and the C₅₋₆ cycloalkyl and 4- to 6-membered heterocyclyl are optionally substituted with one or two substituents each independently selected from the group consisting of oxo, NR¹⁰R¹¹, —C(O)N¹⁰R, and C₁₋₆ alkyl optionally substituted with NR¹⁰R¹¹;
R¹⁰ and R¹¹ are independently H or C₁₋₃ alkyl optionally substituted with C₁₋₆ alkoxy.

In some embodiments, provided is a compound of Formula (III):

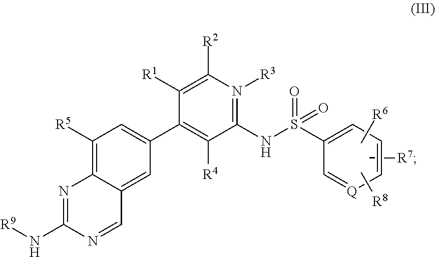

or a stereoisomer, tautomer, or a pharmaceutically acceptable salt thereof, wherein:
R¹, R², R³ and R⁴ are independently selected from the group consisting of H, halo and C₁₋₃ alkyl;
R⁵ is H or C₁₋₃ alkyl;
R⁶, R⁷ and R⁸ are independently selected from the group consisting of H, halo, C₁₋₆ alkyl, and C₁₋₆ alkoxy, wherein the C₁₋₆ alkyl is optionally substituted with OH and the C₁₋₆ alkoxy is optionally substituted with C₁₋₆ alkoxy or NR¹⁰R¹¹;
R⁹ is H, C₁₋₆ alkyl, C₅₋₆ cycloalkyl or 4- to 6-membered heterocyclyl, wherein the C₁₋₆ alkyl is substituted with NR¹⁰R¹¹, and the C₅₋₆ cycloalkyl and 4- to 6-membered heterocyclyl are optionally substituted with one or two substituents each independently selected from the group consisting of oxo, NR¹⁰R¹¹, —C(O)NR¹⁰R¹¹, and C₁₋₆ alkyl optionally substituted with NR¹⁰R¹¹;
R¹⁰ and R¹¹ are independently H or C₁₋₃ alkyl optionally substituted with C₁₋₆ alkoxy.

The compound can be selected from any one of exemplary compounds as shown in Table 1, a stereoisomer, tautomer, or a pharmaceutically acceptable salt thereof.

In some embodiments, the compound is selected from the group consisting of:

| No. | Structure | Name |
|---|---|---|
| 1 | 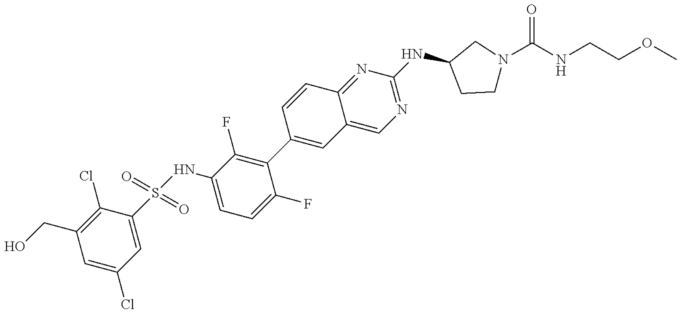 [SMILES: COCCNC(=O)N1CC[C@@H](Nc2ncc3cc(-c4c(F)ccc(NS(=O)(=O)c5cc(Cl)cc(CO)c5Cl)c4F)ccc3n2)C1] | (R)-3-((6-(3- ((2,5-dichloro-3- (hydroxymethyl)phe- nyl)sulfonamido)- 2,6- difluorophenyl)quina- zolin-2- yl)amino)-N-(2- methoxyethyl)pyr- rolidine-1- carboxamide |
| 2 | 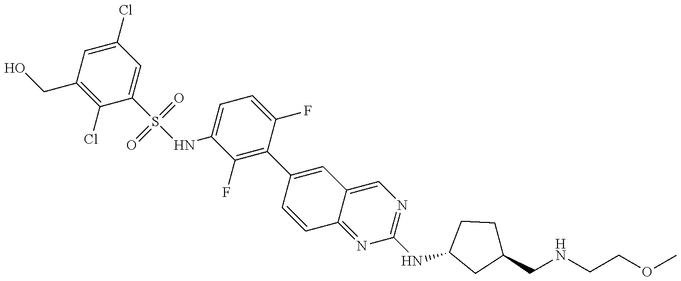 [SMILES: COCCNC[C@@H]1CC[C@@H](Nc2ncc3cc(-c4c(F)ccc(NS(=O)(=O)c5cc(Cl)cc(CO)c5Cl)c4F)ccc3n2)C1] | 2,5-dichloro-N- (2,4-difluoro-3- (2-(((1R,3R)-3- (((2- methoxyethyl)ami- no)methyl)cyclopent- yl)amino)quina- zolin-6- yl)phenyl)-3- (hydroxymethyl)ben- zenesulfonamide |
| 3 | 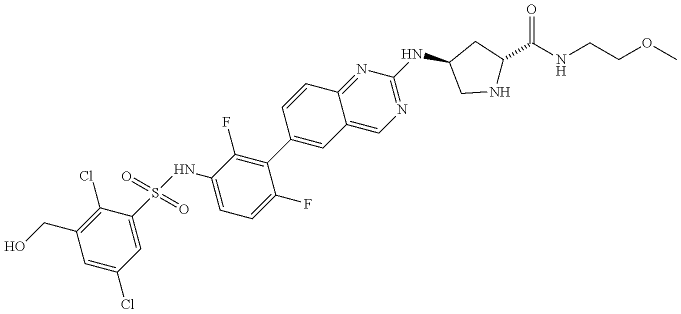 [SMILES: COCCNC(=O)[C@H]1C[C@H](Nc2ncc3cc(-c4c(F)ccc(NS(=O)(=O)c5cc(Cl)cc(CO)c5Cl)c4F)ccc3n2)CN1] | (2R,4S)-4-((6-(3- ((2,5-dichloro-3- (hydroxymethyl)phe- nyl)sulfonamido)- 2,6- difluorophenyl)quin- zolin-2- yl)amino)-N-(2- methoxyethyl)pyr- rolidine-2- carboxamide |
| 4 | 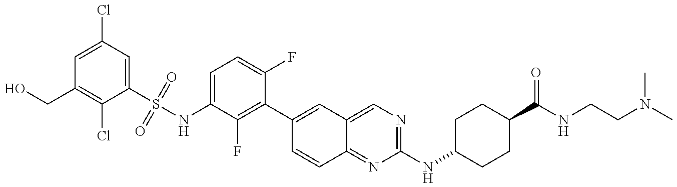 | (1R,4R)-4-((6-(3- ((2,5-dichloro-3- (hydroxymethyl)phe- nyl)sulfonamido)- 2,6- difluorophenyl)quina- zolin-2- yl)amino)-N-(2- (dimethylamino)eth- yl)cyclohexane- 1-carboxamide |
| 5 | 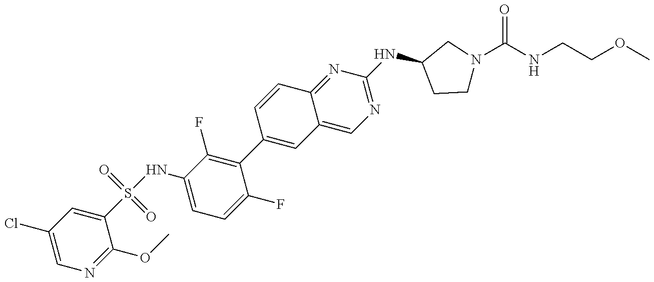 [SMILES: COCCNC(=O)N1CC[C@@H](Nc2ncc3cc(-c4c(F)ccc(NS(=O)(=O)c5cc(Cl)cnc5OC)c4F)ccc3n2)C1] | (R)-3-((6-(3-((5- chloro-2- methoxypyridine)- 3-sulfonamido)- 2,6- difluorophenyl)quina- zolin-2- yl)amino)-N-(2- methoxyethyl)pyr- rolidine-1- carboxamide |
| 6 | 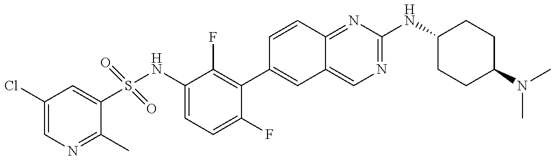 [SMILES: Cc1ncc(Cl)cc1S(=O)(=O)Nc1ccc(F)c(-c2ccc3nc(N[C@H]4CC[C@H](N(C)C)CC4)ncc3c2)c1F] | 5-chloro-N-(3-(2- (((1R,4R)-4- (dimethylamino)cy- clohexyl)amino) quinazolin-6-yl)- 2,4-difluorophenyl)- 2-methylpyridine- 3-sulfonamide |
| 7 | 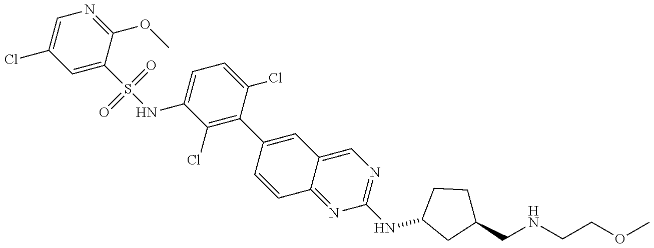 | 5-chloro-N-(2,4- dichloro-3-(2- (((1R,3R)-3-(((2- methoxyethyl)ami- no)methyl)cyclopent- yl)amino)quina- zolin-6- yl)phenyl)-2- methoxypyridine- 3-sulfonamide |
| 8 | 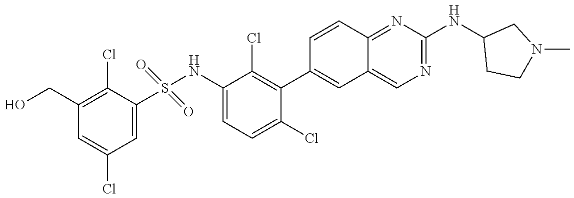 [SMILES: CN1CCC(Nc2ncc3cc(-c4c(Cl)ccc(NS(=O)(=O)c5cc(Cl)cc(CO)c5Cl)c4Cl)ccc3n2)C1] | 2,5-dichloro-N- (2,4-dichloro-3- (2-((1- methylpyrrolidin- 3- yl)amino)quinazo- lin-6-yl)phenyl)- 3- (hydroxymethyl)ben- zenesulfonamide |
| 9 | 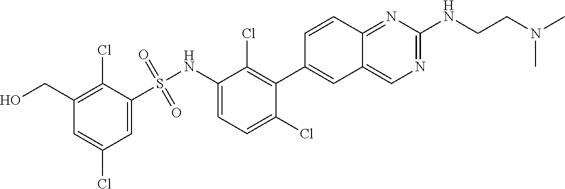 [SMILES: CN(C)CCNc1ncc2cc(-c3c(Cl)ccc(NS(=O)(=O)c4cc(Cl)cc(CO)c4Cl)c3Cl)ccc2n1] | 2,5-dichloro-N- (2,4-dichloro-3- (2-((2- (dimethylamino)eth- yl)amino)quina- zolin-6- yl)phenyl)-3- (hydroxymethyl)ben- zenesulfonamide |
| 10 | 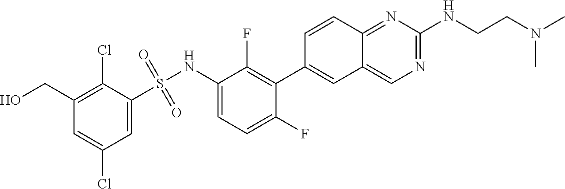 [SMILES: CN(C)CCNc1ncc2cc(-c3c(F)ccc(NS(=O)(=O)c4cc(Cl)cc(CO)c4Cl)c3F)ccc2n1] | 2,5-dichloro-N- (3-(2-((2- (dimethylamino)eth- yl)amino)quina- zolin-6-yl)-2,4- difluorophenyl)- 3- (hydroxymethyl)ben- zenesulfonamide |
| 11 | 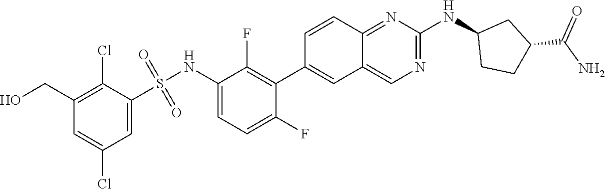 [SMILES: NC(=O)[C@@H]1CC[C@@H](Nc2ncc3cc(-c4c(F)ccc(NS(=O)(=O)c5cc(Cl)cc(CO)c5Cl)c4F)ccc3n2)C1] | (1R,3R)-3-((6-(3- ((2,5-dichloro-3- (hydroxymethyl)phe- nyl)sulfonamido)- 2,6- difluorophenyl)quina- zolin-2- yl)amino)cyclopent- ane-1- carboxamide |
| 12 | 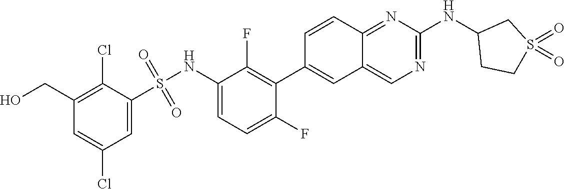 [SMILES: O=S1(=O)CCC(Nc2ncc3cc(-c4c(F)ccc(NS(=O)(=O)c5cc(Cl)cc(CO)c5Cl)c4F)ccc3n2)C1] | 2,5-dichloro-N- (3-(2-((1,1- dioxidotetrahydro- thiophen-3- yl)amino)quinazo- lin-6-yl)-2,4- difluorophenyl)- 3- (hydroxymethyl)ben- zenesulfonamide |
| 13 | 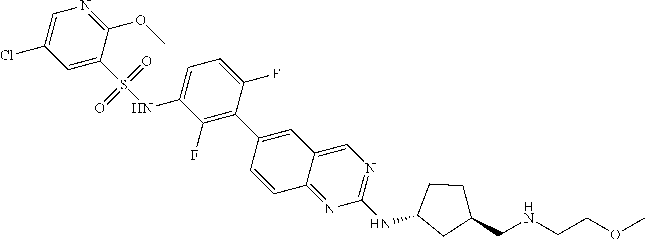 [SMILES: COCCNC[C@@H]1CC[C@@H](Nc2ncc3cc(-c4c(F)ccc(NS(=O)(=O)c5cc(Cl)cnc5OC)c4F)ccc3n2)C1] | 5-chloro-N-(2,4- difluoro-3-(2- (((1R,3R)-3-(((2- methoxyethyl)ami- no)methyl)cyclopent- yl)amino)quina- zolin-6- yl)phenyl)-2- methoxypyridine- 3-sulfonamide |
| 14 | 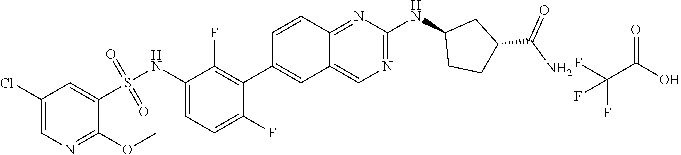 | (1R,3R)-3-((6-(3- ((5-chloro-2- methoxypyridine)- 3-sulfonamido)- 2,6- difluorophenyl)quinaz- zolin-2- yl)amino)cyclopent- ane-1- carboxamide |
| | | 2,2,2- |
| | | trifluoroacetate |
| 15 | 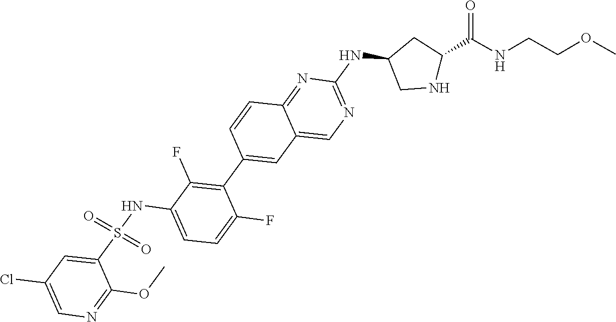 [SMILES: COCCNC(=O)[C@H]1C[C@H](Nc2ncc3cc(-c4c(F)ccc(NS(=O)(=O)c5cc(Cl)cnc5OC)c4F)ccc3n2)CN1] | (2R,4S)-4-((6-(3- ((5-chloro-2- methoxypyridine)- 3-sulfonamido)- 2,6- difluorophenyl)quina- zolin-2- yl)amino)-N-(2- methoxyethyl)pyr- rolidine-2- carboxamide |
| 16 | 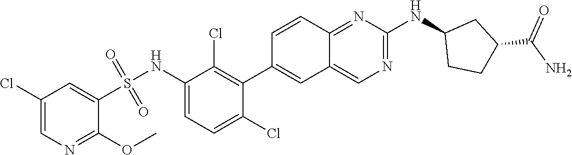 [SMILES: COc1ncc(Cl)cc1S(=O)(=O)Nc1ccc(Cl)c(-c2ccc3nc(N[C@@H]4CC[C@@H](C(N)=O)C4)ncc3c2)c1Cl] | (1R,3R)-3-((6- (2,6-dichloro-3- ((5-chloro-2- methoxypyridine)- 3- sulfonamido)phenyl) quinazolin-2- yl)amino)cyclopent- ane-1- carboxamide |
| 17 | 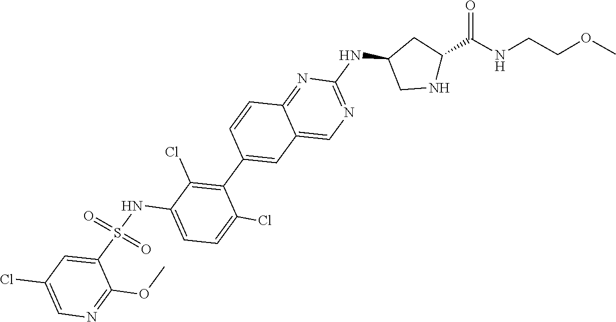 [SMILES: COCCNC(=O)[C@H]1C[C@H](Nc2ncc3cc(-c4c(Cl)ccc(NS(=O)(=O)c5cc(Cl)cnc5OC)c4Cl)ccc3n2)CN1] | (2R,4S)-4-((6- (2,6-dichloro-3- ((5-chloro-2- methoxypyridine)- 3- sulfonamido)phenyl) quinazolin-2- yl)amino)-N-(2- methoxyethyl)pyr- rolidine-2- carboxamide |
| 18 | 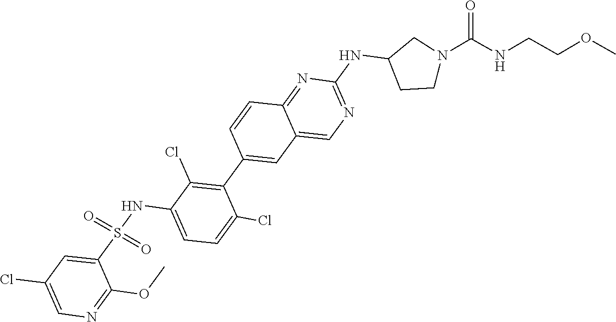 [SMILES: COCCNC(=O)N1CCC(Nc2ncc3cc(-c4c(Cl)ccc(NS(=O)(=O)c5cc(Cl)cnc5OC)c4Cl)ccc3n2)C1] | (R)-3-((6-(2,6- dichloro-3-((5- chloro-2- methoxypyridine)- 3- sulfonamido)phenyl) quinazolin-2- yl)amino)-N-(2- methoxyethyl)pyr- rolidine-2- carboxamide |
| 19 | 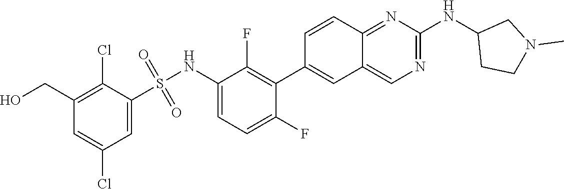 [SMILES: CN1CCC(Nc2ncc3cc(-c4c(F)ccc(NS(=O)(=O)c5cc(Cl)cc(CO)c5Cl)c4F)ccc3n2)C1] | 2,5-dichloro-N- (2,4-difluoro-3- (2-((1- methylpyrrolidin- 3- yl)amino)quinazo- lin-6-yl)phenyl)- 3- (hydroxymethyl)ben- zenesulfonamide |
| 20 | 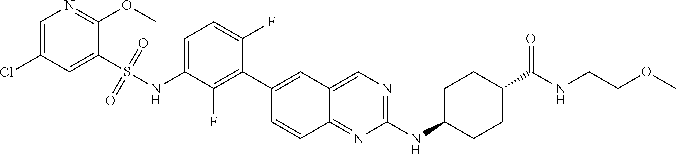 [SMILES: COCCNC(=O)[C@H]1CC[C@H](Nc2ncc3cc(-c4c(F)ccc(NS(=O)(=O)c5cc(Cl)cnc5OC)c4F)ccc3n2)CC1] | (1R,4R)-4-((6-(3- ((5-chloro-2- methoxypyridine)- 3-sulfonamido)- 2,6- difluorophenyl)quina- zolin-2- yl)amino)-N-(2- methoxyethyl)cyclo- hexane-1- |
| | | carboxamide |
| 21 | 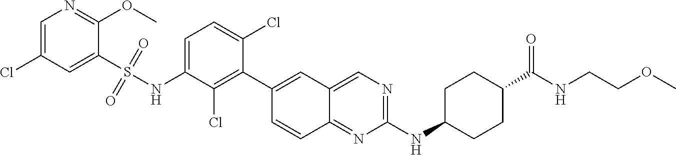 [SMILES: COCCNC(=O)[C@H]1CC[C@H](Nc2ncc3cc(-c4c(Cl)ccc(NS(=O)(=O)c5cc(Cl)cnc5OC)c4Cl)ccc3n2)CC1] | (1R,4R)-4-((6- (2,6-dichloro-3- ((5-chloro-2- methoxypyridine)- 3- sulfonamido)phenyl) quinazolin-2- yl)amino)-N-(2- methoxyethyl)cyclo- hexane-1- |
| | | carboxamide |
| 22 | 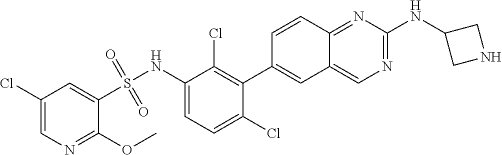 [SMILES: COc1ncc(Cl)cc1S(=O)(=O)Nc1ccc(Cl)c(-c2ccc3nc(NC4CNC4)ncc3c2)c1Cl] | N-(3-(2-(azetidin- 3- ylamino)quinazolin- 6-yl)-2,4- dichlorophenyl)- 5-chloro-2- methoxypyridine- 3-sulfonamide |
| 23 | 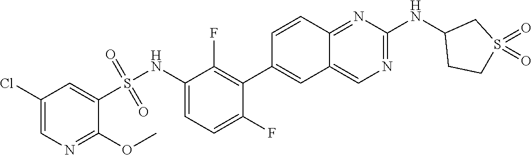 [SMILES: COc1ncc(Cl)cc1S(=O)(=O)Nc1ccc(F)c(-c2ccc3nc(NC4CCS(=O)(=O)C4)ncc3c2)c1F] | 5-chloro-N-(3-(2- ((1,1- dioxidotetrahydro- thiophen-3- yl)amino)quinazo- lin-6-yl)-2,4- difluorophenyl)- 2- methoxypyridine- 3-sulfonamide |
| 27 | 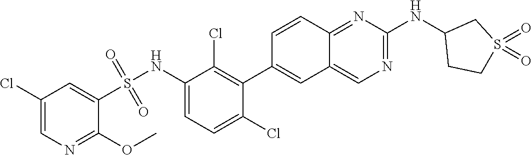 [SMILES: COc1ncc(Cl)cc1S(=O)(=O)Nc1ccc(Cl)c(-c2ccc3nc(NC4CCS(=O)(=O)C4)ncc3c2)c1Cl] | 5-chloro-N-(2,4- dichloro-3-(2- ((1,1- dioxidotetrahydro- thiophen-3- yl)amino)quinazo- lin-6-yl)phenyl)- 2- methoxypyridine- 3-sulfonamide |
| 29 | 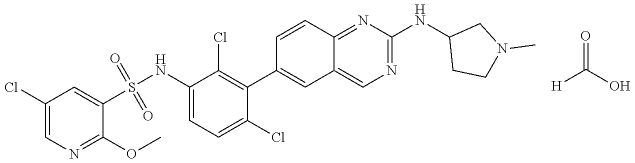 | 5-chloro-N-(2,4- dichloro-3-(2-((1- methylpyrrolidin- 3- yl)amino)quinazo- lin-6-yl)phenyl)- 2- methoxypyridine- 3-sulfonamide formate |
| 30 | 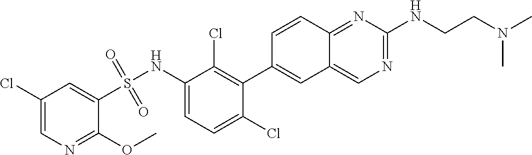 [SMILES: COc1ncc(Cl)cc1S(=O)(=O)Nc1ccc(Cl)c(-c2ccc3nc(NCCN(C)C)ncc3c2)c1Cl] | 5-chloro-N-(2,4- dichloro-3-(2-((2- (dimethylamino)eth- yl)amino)quina- zolin-6- yl)phenyl)-2- methoxypyridine- 3-sulfonamide |
| 33 | 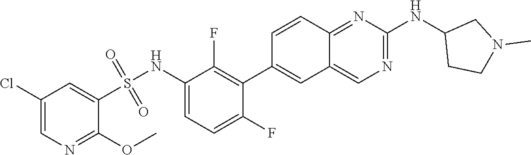 [SMILES: COc1ncc(Cl)cc1S(=O)(=O)Nc1ccc(F)c(-c2ccc3nc(NC4CCN(C)C4)ncc3c2)c1F] | 5-chloro-N-(2,4- difluoro-3-(2-((1- methylpyrrolidin- 3- yl)amino)quinazo- lin-6-yl)phenyl)- 2- methoxypyridine- 3-sulfonamide |
| 34 | 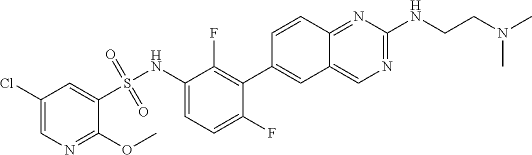 [SMILES: COc1ncc(Cl)cc1S(=O)(=O)Nc1ccc(F)c(-c2ccc3nc(NCCN(C)C)ncc3c2)c1F] | 5-chloro-N-(3-(2- ((2- (dimethylamino)eth- yl)amino)quina- zolin-6-yl)-2,4- difluorophenyl)- 2- methoxypyridine- 3-sulfonamide |
| 35 | 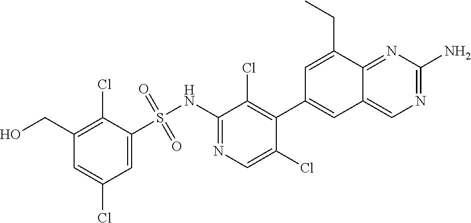 [SMILES: CCc1cc(-c2c(Cl)cnc(NS(=O)(=O)c3cc(Cl)cc(CO)c3Cl)c2Cl)cc2cnc(N)nc12] | N-(4-(2-amino-8- ethylquinazolin- 6-yl)-3,5- dichloropyridin- 2-yl)-2,5- dichloro-3- (hydroxymethyl)ben- zenesulfonamide |
| 38 | 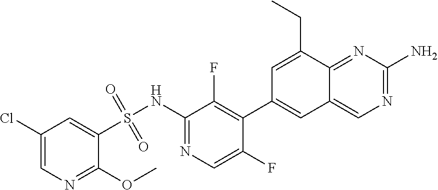 [SMILES: CCc1cc(-c2c(F)cnc(NS(=O)(=O)c3cc(Cl)cnc3OC)c2F)cc2cnc(N)nc12] | N-(4-(2-amino-8- ethylquinazolin- 6-yl)-3,5- difluoropyridin-2- yl)-5-chloro-2- methoxypyridine- 3-sulfonamide |
| 40 | 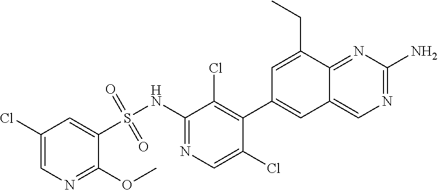 [SMILES: CCc1cc(-c2c(Cl)cnc(NS(=O)(=O)c3cc(Cl)cnc3OC)c2Cl)cc2cnc(N)nc12] | N-(4-(2-amino-8- ethylquinazolin- 6-yl)-3,5- dichloropyridin- 2-yl)-5-chloro-2- methoxypyridine- 3-sulfonamide |
| 45 | 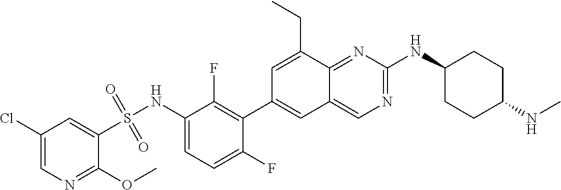 [SMILES: CCc1cc(-c2c(F)ccc(NS(=O)(=O)c3cc(Cl)cnc3OC)c2F)cc2cnc(N[C@H]3CC[C@H](NC)CC3)nc12] | 5-chloro-N-(3-(8- ethyl-2-(((1R,4R)- 4- (methylamino)cyclo- hexyl)amino)quina- zolin-6-yl)- 2,4- difluorophenyl)- 2- methoxypyridine- 3-sulfonamide |
| 46 | 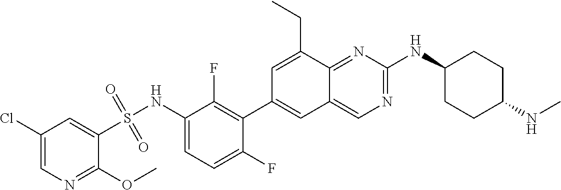 [SMILES: CCc1cc(-c2c(F)ccc(NS(=O)(=O)c3cc(Cl)cnc3OC)c2F)cc2cnc(N[C@H]3CC[C@H](NC)CC3)nc12] | 5-chloro-N-(3-(8- ethyl-2-(((1R,4R)- 4- (methylamino)cyclo- hexyl)amino)quina- zolin-6-yl)- 2,4- difluorophenyl)- 2- methoxypyridine- 3-sulfonamide |
| 47 | 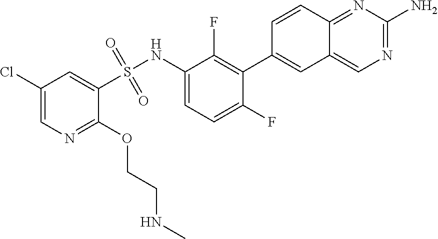 [SMILES: CNCCOc1ncc(Cl)cc1S(=O)(=O)Nc1ccc(F)c(-c2ccc3nc(N)ncc3c2)c1F] | N-(3-(2- aminoquinazolin- 6-yl)-2,4- difluorophenyl)- 5-chloro-2-(2- (methylamino)eth- oxy)pyridine- sulfonamide |
| 48 | 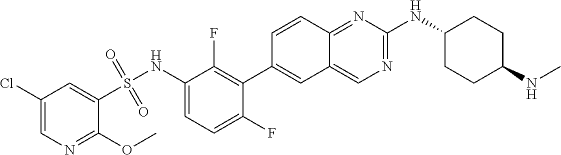 [SMILES: CN[C@H]1CC[C@H](Nc2ncc3cc(-c4c(F)ccc(NS(=O)(=O)c5cc(Cl)cnc5OC)c4F)ccc3n2)CC1] | 5-chloro-N-(2,4- difluoro-3-(2- (((1R,4R)-4- (methylamino)cyclo- hexyl)amino)quina- zolin-6- yl)phenyl)-2- methoxypyridine- 3-sulfonamide |
| 49 | 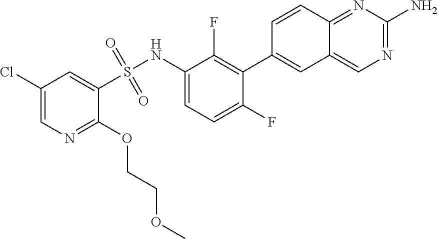 [SMILES: COCCOc1ncc(Cl)cc1S(=O)(=O)Nc1ccc(F)c(-c2ccc3nc(N)ncc3c2)c1F] | N-(3-(2- aminoquinazolin- 6-yl)-2,4- difluorophenyl)- 5-chloro-2-(2- methoxyethoxy)py- ridine-3- sulfonamide |
| 51 | 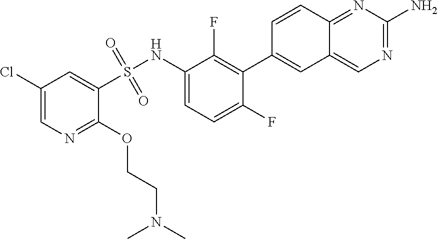 [SMILES: CN(C)CCOc1ncc(Cl)cc1S(=O)(=O)Nc1ccc(F)c(-c2ccc3nc(N)ncc3c2)c1F] | N-(3-(2- aminoquinazolin- 6-yl)-2,4- difluorophenyl)- 5-chloro-2-(2- (dimethylamino)eth- oxy)pyridine-3- sulfonamide |
| 52 | 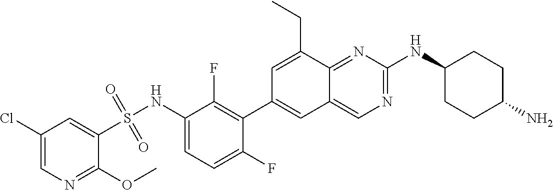 [SMILES: CCc1cc(-c2c(F)ccc(NS(=O)(=O)c3cc(Cl)cnc3OC)c2F)cc2cnc(N[C@H]3CC[C@H](N)CC3)nc12] | N-(3-(2- (((1R,4R)-4- aminocyclohexyl) amino)-8- ethylquinazolin- 6-yl)-2,4- difluorophenyl)- 5-chloro-2- methoxypyridine- 3-sulfonamide |
| 53 | 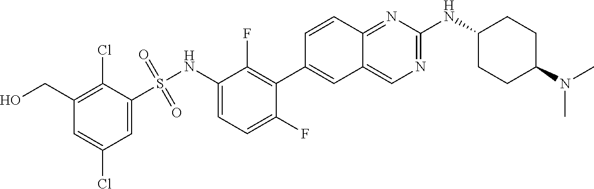 [SMILES: CN(C)[C@H]1CC[C@H](Nc2ncc3cc(-c4c(F)ccc(NS(=O)(=O)c5cc(Cl)cc(CO)c5Cl)c4F)ccc3n2)CC1] | 2,5-dichloro-N- (3-(2-(((1R,4R)-4- (dimethylamino)cy- clohexyl)amino) quinazolin-6-yl)- 2,4-difluorophenyl)- 3- (hydroxymethyl)ben- zenesulfonamide |
| 54 | 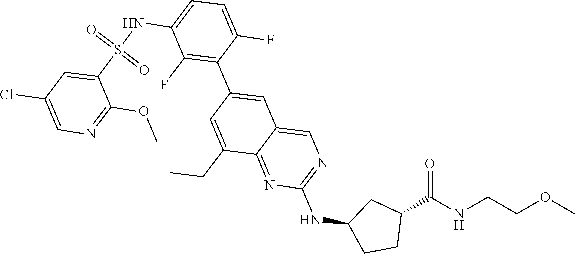 [SMILES: CCc1cc(-c2c(F)ccc(NS(=O)(=O)c3cc(Cl)cnc3OC)c2F)cc2cnc(N[C@@H]3CC[C@@H](C(=O)NCCOC)C3)nc12] | (1R,3R)-3-((6-(3- ((5-chloro-2- methoxypyridine)- 3-sulfonamido)- 2,6- difluorophenyl)- 8- ethylquinazolin- 2-yl)amino)-N-(2- methoxyethyl)cyclo- pentane-1- carboxamide |
| 57 | 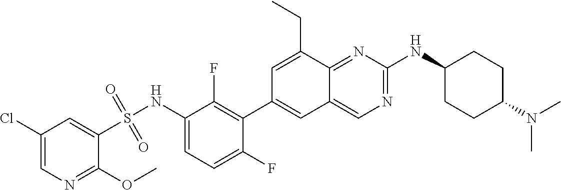 [SMILES: CCc1cc(-c2c(F)ccc(NS(=O)(=O)c3cc(Cl)cnc3OC)c2F)cc2cnc(N[C@H]3CC[C@H](N(C)C)CC3)nc12] | 5-chloro-N-(3-(2- (((1R,4R)-4- (dimethylamino)cy- clohexyl)amino)- 8- ethylquinazolin- 6-yl)-2,4- difluorophenyl)- 2- methoxypyridine- 3-sulfonamide |
| 61 | 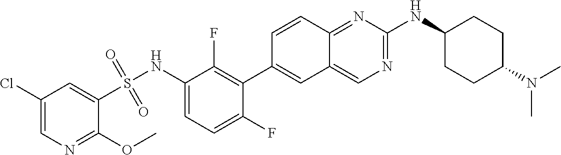 [SMILES: COc1ncc(Cl)cc1S(=O)(=O)Nc1ccc(F)c(-c2ccc3nc(N[C@H]4CC[C@H](N(C)C)CC4)ncc3c2)c1F] | 5-chloro-N-(3-(2- (((1R,4R)-4- (dimethylamino)cy- clohexyl)amino) quinazolin-6-yl)- 2,4- difluorophenyl)- 2- methoxypyridine- 3-sulfonamide |
| 62 | 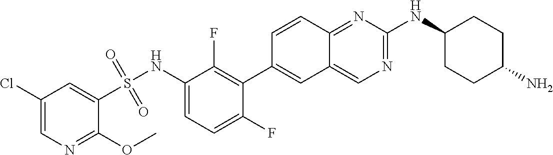 [SMILES: COc1ncc(Cl)cc1S(=O)(=O)Nc1ccc(F)c(-c2ccc3nc(N[C@H]4CC[C@H](N)CC4)ncc3c2)c1F] | N-(3-(2- (((1R,4R)-4- aminocyclohexyl) amino)quinazolin- 6-yl)-2,4- difluorophenyl)- 5-chloro-2- methoxypyridine- 3-sulfonamide |
| 64 | 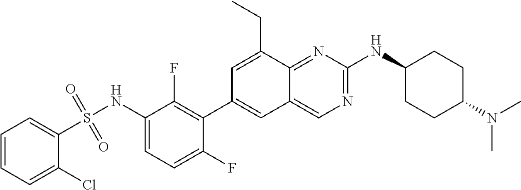 [SMILES: CCc1cc(-c2c(F)ccc(NS(=O)(=O)c3ccccc3Cl)c2F)cc2cnc(N[C@H]3CC[C@H](N(C)C)CC3)nc12] | 2-chloro-N-(3-(2- (((1R,4R)-4- (dimethylamino)cy- clohexyl)amino)- 8- ethylquinazolin- 6-yl)-2,4- difluorophenyl)ben- zenesulfonamide |
| 65 | 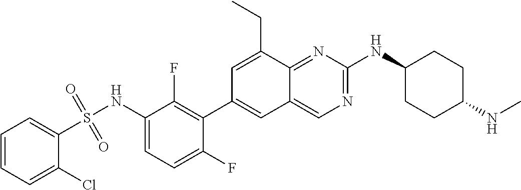 [SMILES: CCc1cc(-c2c(F)ccc(NS(=O)(=O)c3ccccc3Cl)c2F)cc2cnc(N[C@H]3CC[C@H](NC)CC3)nc12] | 2-chloro-N-(3-(8- ethyl-2-(((1R,4R)- 4- (methylamino)cyclo- hexyl)amino)quina- zolin-6-yl)- 2,4- difluorophenyl)ben- zenesulfonamide |
| 66 | 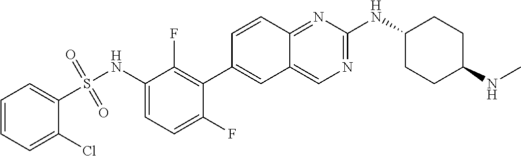 [SMILES: CN[C@H]1CC[C@H](Nc2ncc3cc(-c4c(F)ccc(NS(=O)(=O)c5ccccc5Cl)c4F)ccc3n2)CC1] | 2-chloro-N-(2,4- difluoro-3-(2- (((1R,4R)-4- (methylamino)cyclo- hexyl)amino)quina- zolin-6- yl)phenyl)benzene- sulfonamide |
| 68 | 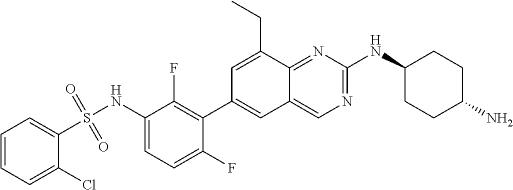 [SMILES: CCc1cc(-c2c(F)ccc(NS(=O)(=O)c3ccccc3Cl)c2F)cc2cnc(N[C@H]3CC[C@H](N)CC3)nc12] | N-(3-(2- (((1R,4R)-4- aminocyclohexyl) amino)-8- ethylquinazolin- 6-yl)-2,4- difluorophenyl)- 2- chlorobenzenesulfon- amide |
| 70 | 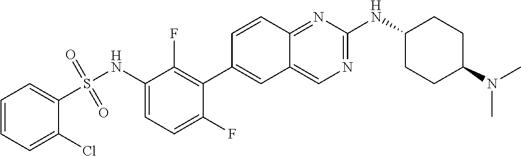 [SMILES: CN(C)[C@H]1CC[C@H](Nc2ncc3cc(-c4c(F)ccc(NS(=O)(=O)c5ccccc5Cl)c4F)ccc3n2)CC1] | 2-chloro-N-(3-(2- (((1R,4R)-4- (dimethylamino)cy- clohexyl)amino) quinazolin-6-yl)- 2,4- difluorophenyl)ben- zenesulfonamide |
| 71 | 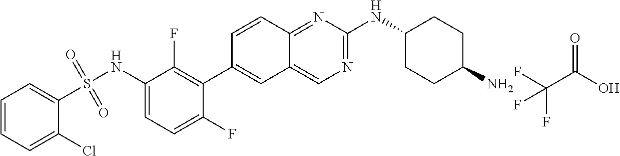 | N-(3-(2- (((1R,4R)-4- aminocyclohexyl) amino)quinazolin- 6-yl)-2,4- difluorophenyl)- 2- chlorobenzenesulfon- amide 2,2,2- trifluoroacetate |
| 73 | 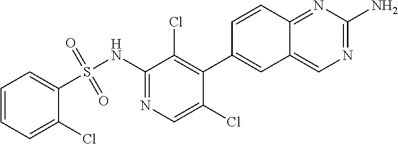 [SMILES: Nc1ncc2cc(-c3c(Cl)cnc(NS(=O)(=O)c4ccccc4Cl)c3Cl)ccc2n1] | N-(4-(2- aminoquinazolin- 6-yl)-3,5- dichloropyridin- 2-yl)-2- chlorobenzenesulfon- amide |
| 75 | 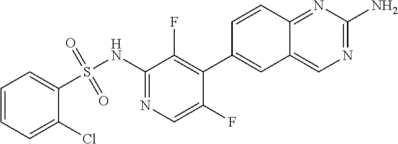 [SMILES: Nc1ncc2cc(-c3c(F)cnc(NS(=O)(=O)c4ccccc4Cl)c3F)ccc2n1] | N-(4-(2- aminoquinazolin- 6-yl)-3,5- difluoropyridin-2- yl)-2- chlorobenzenesulfon- amide |
| 78 | 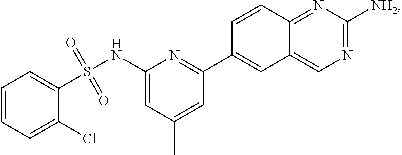 [SMILES: Cc1cc(NS(=O)(=O)c2ccccc2Cl)nc(-c2ccc3nc(N)ncc3c2)c1] | N-(6-(2- aminoquinazolin- 6-yl)-4- methylpyridin-2- yl)-2- chlorobenzenesulfon- amide |
| | and | |
| 82 | 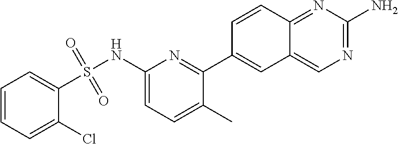 [SMILES: Cc1ccc(NS(=O)(=O)c2ccccc2Cl)nc1-c1ccc2nc(N)ncc2c1] | N-(6-(2- aminoquinazolin- 6-yl)-5- methylpyridin-2- yl)-2- chlorobenzenesulfon- amide |

or a stereoisomer, tautomer, or a pharmaceutically acceptable salt thereof.

In another aspect, provided is a compound of Formula (Ia):

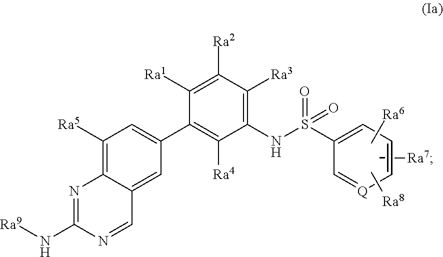

or a stereoisomer, tautomer, or a pharmaceutically acceptable salt thereof, wherein:
Ra¹ and Ra⁴ are halo;
Ra² and Ra³ are independently selected from the group consisting of H, halo and C₁₋₃ alkyl;
Ra⁵ is H or C₁₋₃ alkyl;
Ra⁶, R⁷ and R⁸ are independently selected from the group consisting of H, halo, C₁₋₆ alkyl, and C₁₋₆ alkoxy, wherein the C₁₋₆ alkyl and C₁₋₆ alkoxy are optionally substituted with OH;
Ra⁹ is H, C₅₋₆ cycloalkyl or 5 or 6-membered heterocyclyl, wherein the C₅₋₆ cycloalkyl and 5 or 6-membered heterocyclyl are optionally substituted with OH; and
Q is C, CH or N.

In the descriptions herein, it is understood that every description, variation, embodiment, or aspect of a moiety may be combined with every description, variation, embodiment, or aspect of other moieties the same as if each and every combination of descriptions is specifically and individually listed.

In some embodiments, Ra² and Ra³ are H.

In some embodiments, Ra⁵ is C₁₋₃ alkyl (e.g., methyl and ethyl).

In some embodiments, at least one of Ra⁶, Ra⁷ and Ra⁸ is not H. In some embodiments, the moiety:
is selected from the group consisting of:

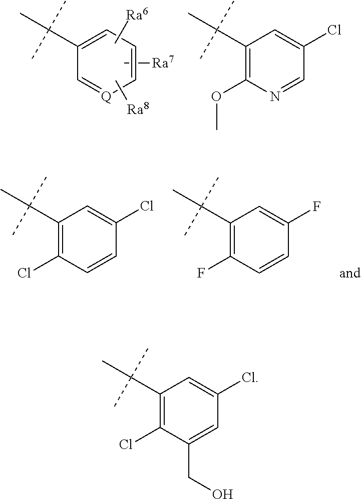

In some embodimentns, the moiety is

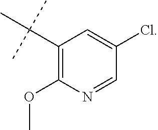

In some embodiments, Ra⁹ is H. In some embodiments, Ra⁹ is C₅₋₆ cycloalkyl or 5 or 6-membered heterocyclyl, wherein the C₅₋₆ cycloalkyl and 5 or 6-membered heterocyclyl are optionally substituted with OH. In some embodiments, Ra⁹ is

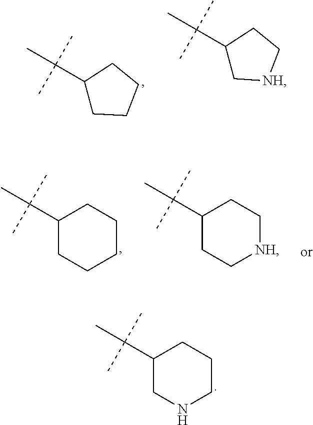

In some embodiments, Ra⁹ is H and Q is N.

In some embodiments, provided is a compound of formula (IIa):

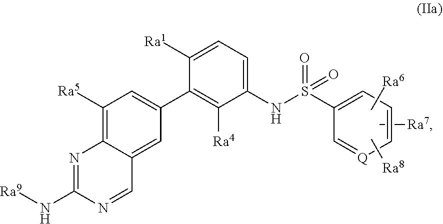

or a stereoisomer, tautomer, or a pharmaceutically acceptable salt thereof, wherein:
Ra¹ and Ra⁴ are halo;
Ra⁵ is H or C₁₋₃ alkyl;
Ra⁶, Ra⁷ and Ra⁸ are independently selected from the group consisting of H, halo, C₁₋₆ alkyl, and C₁₋₆ alkoxy, wherein the C₁₋₆ alkyl and C₁₋₆ alkoxy are optionally substituted with OH;
Ra⁹ is

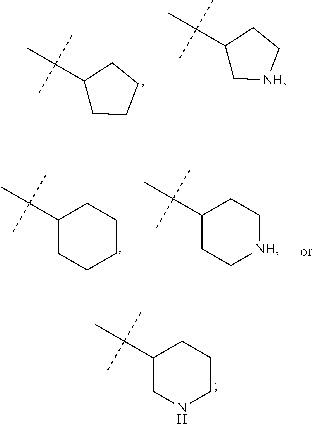

and
Q is C, CH or N.

In some embodiments, provided is a compound of formula (IIIa):

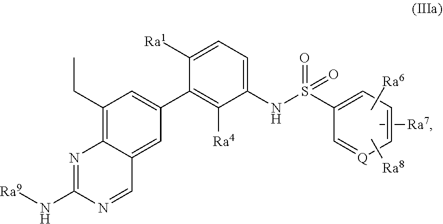

or a stereoisomer, tautomer, or a pharmaceutically acceptable salt thereof, wherein:
Ra¹ and Ra⁴ are halo;
Ra⁶, Ra⁷ and Ra⁸ are independently selected from the group consisting of H, halo, C₁₋₆ alkyl, and C₁₋₆ alkoxy, wherein the C₁₋₆ alkyl and C₁₋₆ alkoxy are optionally substituted with OH;
Ra⁹ is H, C₅₋₆ cycloalkyl or 5 or 6-membered heterocyclyl, wherein the C₅₋₆ cycloalkyl and 5 or 6-membered heterocyclyl are optionally substituted with OH; and
Q is C, CH or N.

Some compounds of Formula (Ia) and derivatives or synthetic intermediates thereof can be prepared in accordance with synthetic methods known to the skilled person. In some embodiments, the invention provides a process for the preparation of a compound as defined in Formula (Ia) above. Certain compounds of the invention may be prepared according to the methods described in WO 2021/165346.

For GCN2 enzyme assays, testing compounds were prepared at proper concentrations in DMSO solution. IC₅₀ values were calculated from a remaining activity using read conversion ratio.

The compound can be selected from any one of exemplary compounds as shown in Table 1a, a stereoisomer, tautomer, or a pharmaceutically acceptable salt thereof.

In some embodiments, the compound is selected from the group consisting of:

| No. | Structure | Name |
|---|---|---|
| 24a | 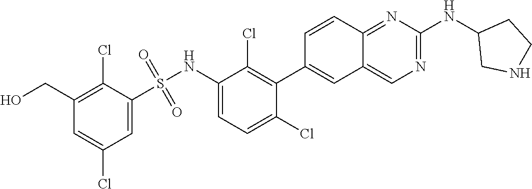 [SMILES: O=S(=O)(Nc1ccc(Cl)c(-c2ccc3nc(NC4CCNC4)ncc3c2)c1Cl)c1cc(Cl)cc(CO)c1Cl] | 2,5-dichloro-N-(2,4-dichloro- 3-(2-(pyrrolidin-3- ylamino)quinazolin-6- yl)phenyl)-3- (hydroxymethyl)benzenesulfon- amide |
| 25a | 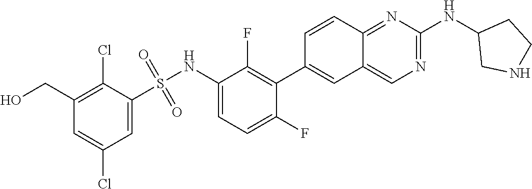 [SMILES: O=S(=O)(Nc1ccc(F)c(-c2ccc3nc(NC4CCNC4)ncc3c2)c1F)c1cc(Cl)cc(CO)c1Cl] | 2,5-dichloro-N-(2,4-difluoro- 3-(2-(pyrrolidin-3- ylamino)quinazolin-6- yl)phenyl)-3- (hydroxymethyl)benzensulfon- amide |
| 26a | 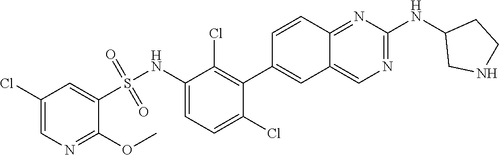 [SMILES: COc1ncc(Cl)cc1S(=O)(=O)Nc1ccc(Cl)c(-c2ccc3nc(NC4CCNC4)ncc3c2)c1Cl] | 5-chloro-N-(2,4-dichloro-3- (2-(pyrrolidin-3- ylamino)quinazolin-6- yl)phenyl)-2- methoxypyridine-3- sulfonamide |
| 28a | 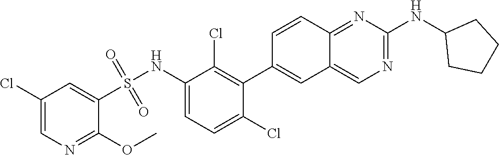 [SMILES: COc1ncc(Cl)cc1S(=O)(=O)Nc1ccc(Cl)c(-c2ccc3nc(NC4CCCC4)ncc3c2)c1Cl] | 5-chloro-N-(2,4-dichloro-3- (2- (cyclopentylamino)quinazolin- 6-yl)phenyl)-2- methoxpyyridine-3- sulfonamide |
| 31a | 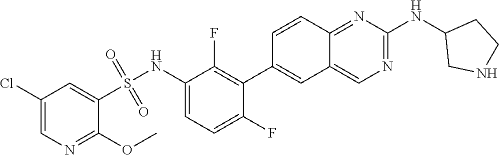 [SMILES: COc1ncc(Cl)cc1S(=O)(=O)Nc1ccc(F)c(-c2ccc3nc(NC4CCNC4)ncc3c2)c1F] | 5-chloro-N-(2,4-difluoro-3-(2- (pyrrolidin-3- ylamino)quinazolin-6- yl)phenyl)-2- methoxypyridine-3- sulfonamide |
| 32a | 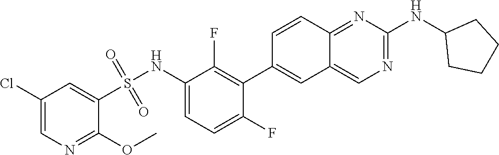 [SMILES: COc1ncc(Cl)cc1S(=O)(=O)Nc1ccc(F)c(-c2ccc3nc(NC4CCCC4)ncc3c2)c1F] | 5-chloro-N-(3-(2- (cyclopentylamino)quinazolin- 6-yl)-2,4-difluorophenyl)-2- methoxypridine-3- sulfonamide |
| 36a | 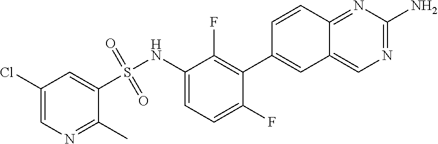 [SMILES: Cc1ncc(Cl)cc1S(=O)(=O)Nc1ccc(F)c(-c2ccc3nc(N)ncc3c2)c1F] | N-(3-(2-aminoquinazolin-6- yl)-2,4-difluorophenyl)-5- chloro-2-methylpyridine-3- sulfonamide |
| 37a | 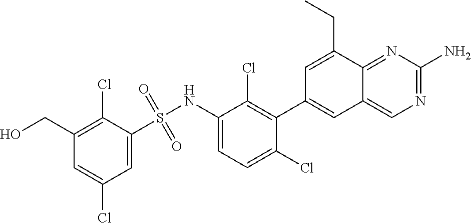 [SMILES: CCc1cc(-c2c(Cl)ccc(NS(=O)(=O)c3cc(Cl)cc(CO)c3Cl)c2Cl)cc2cnc(N)nc12] | N-(3-(2-amino-8- ethylquinazolin-6-yl)-2,4- dichlorophenyl)-2,5-dichloro- 3- (hydroxymethyl)benzenesulfon- amide |
| 39a | 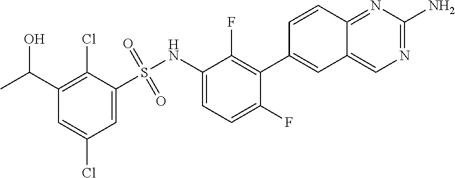 [SMILES: CC(O)c1cc(Cl)cc(S(=O)(=O)Nc2ccc(F)c(-c3ccc4nc(N)ncc4c3)c2F)c1Cl] | N-(3-(2-aminoquinazolin-6- yl)-2,4-difluorophenyl)-2,5- dichloro-3-(1- hydroxyethyl)benzenesulfona- mide |
| 41a | 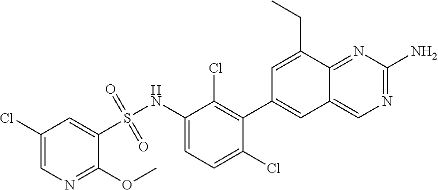 [SMILES: CCc1cc(-c2c(Cl)ccc(NS(=O)(=O)c3cc(Cl)cnc3OC)c2Cl)cc2cnc(N)nc12] | N-(3-(2-amino-8- ethylquinazolin-6-yl)-2,4- dichlorophenyl)--chloro-2- methoxypyridine-3- sulfonamide |
| 42a | 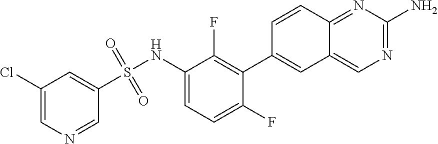 [SMILES: Nc1ncc2cc(-c3c(F)ccc(NS(=O)(=O)c4cncc(Cl)c4)c3F)ccc2n1] | N-(3-(2-aminoquinazolin-6- yl)-2,4-difluorophenyl)-5- chloropyridine-3-sulfonamide |
| 43a | 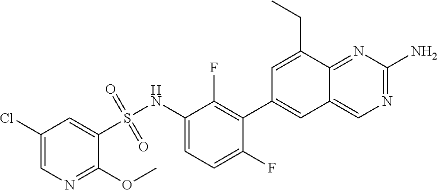 [SMILES: CCc1cc(-c2c(F)ccc(NS(=O)(=O)c3cc(Cl)cnc3OC)c2F)cc2cnc(N)nc12] | N-(3-(2-amino-8- ethylquinazolin-6-yl)-2,4- difluorophenyl)-5-chloro-2- methoxypyridine-3- sulfonamide |
| 44a | 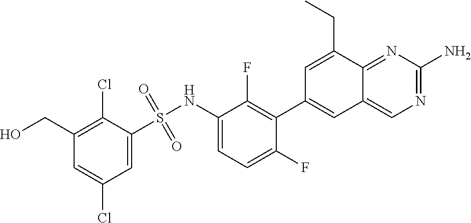 [SMILES: CCc1cc(-c2c(F)ccc(NS(=O)(=O)c3cc(Cl)cc(CO)c3Cl)c2F)cc2cnc(N)nc12] | N-(3-(2-amino-8- ethylquinazolin-6-yl)-2,4- difluorophenyl)-2,5-dichloro- 3- (hydroxymethyl)benzenesulfon- amide |
| 50a | 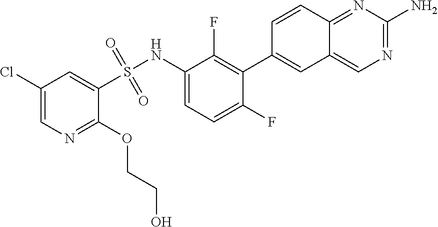 [SMILES: Nc1ncc2cc(-c3c(F)ccc(NS(=O)(=O)c4cc(Cl)cnc4OCCO)c3F)ccc2n1] | N-(3-(2-aminoquinazolin-6- yl)-2,4-difluorophenyl)-5- chloro-2-(2- hydroxyethoxy)pyridine-3- sulfonamide |
| 55a | 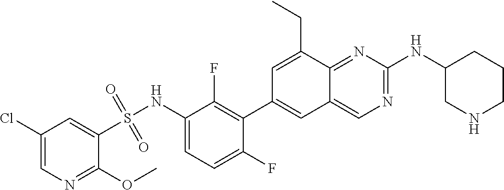 [SMILES: CCc1cc(-c2c(F)ccc(NS(=O)(=O)c3cc(Cl)cnc3OC)c2F)cc2cnc(NC3CCCNC3)nc12] | 5-chloro-N-(3-(8-ethyl-2- (piperidin-3- ylamino)quinazolin-6-yl)-2,4- difluorophenyl)-2- methoxypyridine-3- sulfonamide |
| 56a | 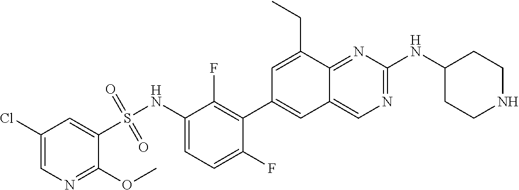 [SMILES: CCc1cc(-c2c(F)ccc(NS(=O)(=O)c3cc(Cl)cnc3OC)c2F)cc2cnc(NC3CCNCC3)nc12] | 5-chloro-N-(3-(8-ethyl-2- (piperidin-4- ylamino)quinazolin-6-yl)-2,4- difluoropehnyl)-2- methoxypyridine-3- sulfonamide |
| 58a | 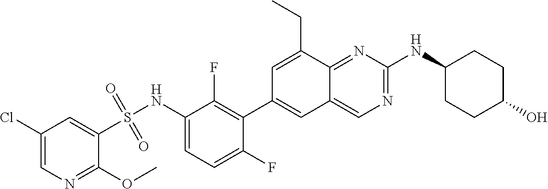 [SMILES: CCc1cc(-c2c(F)ccc(NS(=O)(=O)c3cc(Cl)cnc3OC)c2F)cc2cnc(N[C@H]3CC[C@H](O)CC3)nc12] | 5-chloro-N-(3-(8-ethyl-2- (((1R,4R)-4- hydroxycyclohexyl)amino) quinazolin-6-yl)-2,4- difluorophenyl)-2- methoxypyridine-3- sulfonamide |
| 59a | 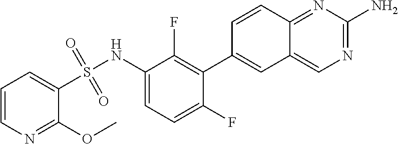 [SMILES: COc1ncccc1S(=O)(=O)Nc1ccc(F)c(-c2ccc3nc(N)ncc3c2)c1F] | N-(3-(2-aminoquinazolin-6- yl)-2,4-difluorophenyl)-2- methoxypyridine-3- sulfonamide |
| 60a | 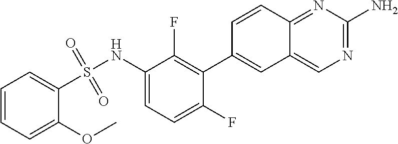 [SMILES: COc1ccccc1S(=O)(=O)Nc1ccc(F)c(-c2ccc3nc(N)ncc3c2)c1F] | N-(3-(2-aminoquinazolin-6- yl)-2,4-difluorophenyl)-2- methoxybenzenesulfonamide |
| 63a | 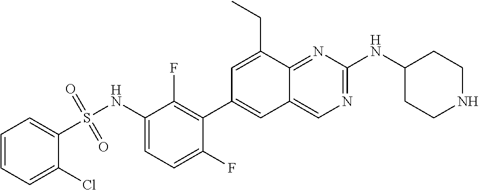 [SMILES: CCc1cc(-c2c(F)ccc(NS(=O)(=O)c3ccccc3Cl)c2F)cc2cnc(NC3CCNCC3)nc12] | 2-chloro-N-(3-(8-ethyl-2- (piperidin-4- ylamino)quinazolin-6-yl)-2,4- difluorophenyl)benzenesulfon- amide |
| 67a | 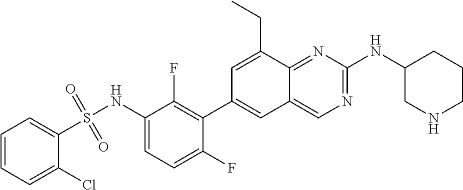 [SMILES: CCc1cc(-c2c(F)ccc(NS(=O)(=O)c3ccccc3Cl)c2F)cc2cnc(NC3CCCNC3)nc12] | 2-chloro-N-(3-(8-ethyl-2- (piperidin-3- ylamino)quinazolin-6-yl)-2,4- difluorophenyl)benzenesulfon- amide |
| 69a | 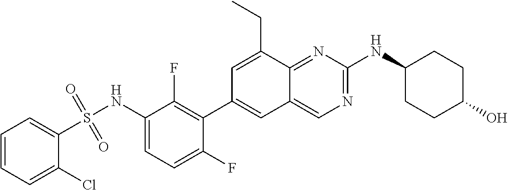 [SMILES: CCc1cc(-c2c(F)ccc(NS(=O)(=O)c3ccccc3Cl)c2F)cc2cnc(N[C@H]3CC[C@H](O)CC3)nc12] | 2-chloro-N-(3-(8-ethyl-2- (((1R,4R)-4- hydroxycyclohexyl)amino)quina- zolin-6-yl)-2,4- difluorophenyl)benzenesulfon- amide |
| 72a | 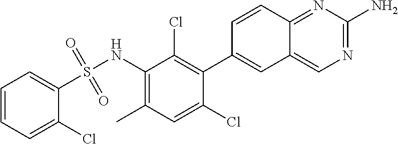 [SMILES: Cc1cc(Cl)c(-c2ccc3nc(N)ncc3c2)c(Cl)c1NS(=O)(=O)c1ccccc1Cl] | N-(3-(2-aminoquinazolin-6- yl)-2,4-dichloro-6- methylphenyl)-2- chlorobenzenesulfonamide |
| 74a | 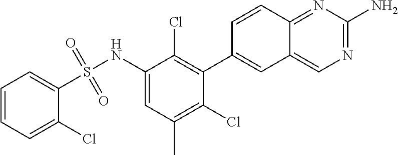 [SMILES: Cc1cc(NS(=O)(=O)c2ccccc2Cl)c(Cl)c(-c2ccc3nc(N)ncc3c2)c1Cl] | N-(3-(2-aminoquinazolin-6- yl)-2,4-dichloro-5- methylphenyl)-2- chlorobenzenesulfonamide |
| 76a | 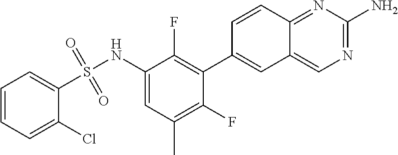 [SMILES: Cc1cc(NS(=O)(=O)c2ccccc2Cl)c(F)c(-c2ccc3nc(N)ncc3c2)c1F] | N-(3-(2-aminoquinazolin-6- yl)-2,4-difluoro-5- methylphenyl)-2- chlorobenzenesulfonamide |
| 77a | 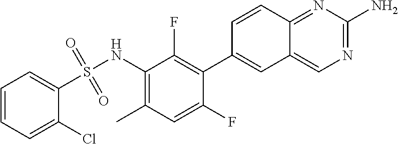 [SMILES: Cc1cc(F)c(-c2ccc3nc(N)ncc3c2)c(F)c1NS(=O)(=O)c1ccccc1Cl] | N-(3-(2-aminoquinazolin-6- yl)-2,4-difluoro-6- methylphenyl)-2- chlorobenzenesulfonamide |
| 79a | 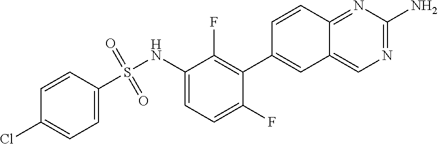 [SMILES: Nc1ncc2cc(-c3c(F)ccc(NS(=O)(=O)c4ccc(Cl)cc4)c3F)ccc2n1] | N-(3-(2-aminoquinazolin-6- yl)-2,4-difluorophenyl)-4- chlorobenzenesulfonamide |
| 80a | 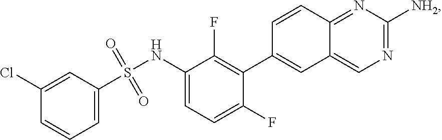 [SMILES: Nc1ncc2cc(-c3c(F)ccc(NS(=O)(=O)c4cccc(Cl)c4)c3F)ccc2n1] | N-(3-(2-aminoquinazolin-6- yl)-2,4-difluorophenyl)-3- chlorobenzenesulfonamide |
| | and | |
| 81a | 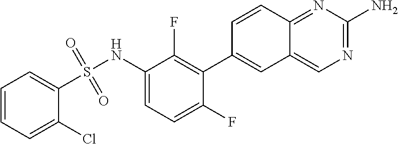 [SMILES: Nc1ncc2cc(-c3c(F)ccc(NS(=O)(=O)c4ccccc4Cl)c3F)ccc2n1] | N-(3-(2-aminoquinazolin-6- yl)-2,4-difluorophenyl)-2- chlorobenzenesulfonamide |

or a stereoisomer, tautomer, or a pharmaceutically acceptable salt thereof.

### Methods of Treatment

Further embodiments of the invention include the use of a compound of Formula (1) or a salt thereof or a pharmaceutical composition comprising a compound of Formula (1) as a GCN2 modulator. Compounds of the present invention may be used as GCN2 modulators. Compounds of the present invention may be used as GCN2 inhibitors, antagonists or agonists. Compounds of the present invention may be used in the treatment of a disease or disorder characterised by activation of GCN2.

Compounds of the present invention may be used in the treatment of cancer, neurodegenerative diseases, chronic infections or conditions or symptoms related thereto.

Compounds of the present invention may be used in the treatment of cancer. In some embodiments, compounds of the present invention are used to treat breast cancer, colorectal cancer, ovarian cancer, prostate cancer, pancreatic cancer, kidney cancer, lung cancer, melanoma, fibrosarcoma, bone sarcoma, connective tissue sarcoma, renal cell carcinoma, giant cell carcinoma, squamous cell carcinoma, leukemia, skin cancer, soft tissue cancer, liver cancer, gastrointestinal carcinoma, adenocarcinoma, hepatocellular carcinoma, thyroid cancer, multiple myeloma, cancer of secretory cells, myelodysplastic syndrome, myeloproliferative neoplasm, malignant glioma, non-Hodgkin's lymphoma, Hodgkin's lymphoma, Burkitt's lymphoma, chronic lymphocytic leukemia, chronic myeloid leukemia, hairy cell leukemia, monoclonal gammopathy of undetermined significance (MGUS), plasmacytoma, lymphoplasmacytic lymphoma, acute lymphoblastic leukemia, acute myeloid leukemia, chronic myelomonocytic leukemia, juvenile myelomonocytic leukemia, large granular lymphocytic leukemia, B-cell prolymphocytic leukemia, T-cell prolymphocytic leukemia. small cell lung cancer, malignant pleural mesothelioma, Head and neck squamous cell carcinoma, glioblastoma multiforme, sarcoma, pediatric neuroblastoma or conditions or symptoms related thereto.

In some embodiments, compounds and compositions detailed herein are used as modulators of GCN2. Provided herein is a method of treating a disease in an individual comprising administering an effective amount of a compound of Formula (I) and Formula (Ia) or any embodiment, variation or aspect thereof.

It was demonstrated that GCN2 may be used as an immune regulator. GCN2 senses tryptophan and L-arginine deficiency. Indoleamine 2,3-dioxygenase (IDO) is a potent immunoregulatory enzyme that mediates conversion of the essential amino acid tryptophan (Trp) to kyneurines and its expression allows certain macrophages and dendritic cells (DCs) to inhibit T cell proliferation (Grohmann et al., 2003, Mellor and Munn, 2004). IDO and the subsequent tryptophan starvation triggers a GCN2-dependent stress signalling pathway that induced profound anergy in responding T cells. GCN2-knockout cells are refractory to IDO-induced cell cycle arrest and anergy. IDO-expressing plasmacytoid DCs are found in tumor-draining lymph nodes and activates the GCN2 kinase pathway in responding T cells. T cells with a targeted disruption of GCN2 are not susceptible to IDO-mediated suppression of proliferation. (Munn et al., 2005). GCN2 activation through accumulation of unloaded tRNAs leads to inhibition of CD8+ effector T-cells and increase in generation and activation of regulatory T-cells.

IFN-γ is a cytokine that is produced mostly by activated T cells and NK cells and has complex effects on immune and nonimmune cells. IFN-γ plays important roles in inflammation, making it particularly relevant to transplantation and autoimmune diseases. IFN-γ induces Trp metabolism, which then activates GCN2 kinase, leading to the phosphorylation of the eIF2α, an activator of autophagy. Conversely, Trp supplementation reduces the activation of the GCN2-eIF2α pathway and inhibits autophagy. Further, targeting of GCN2 expression by RNA interference also inhibits IFN-γ-induced autophagy (Fougeray et al., 2012).

GCN2 plays a central role in regulating the cell-cycle arrest induced by L-Arginine (L-Arg) starvation. L-Arg is a nonessential amino acid that plays a central role in regulating the immune response (Bronte and Zanovello, 2005). In tumor-infiltrating myeloid cells, L-Arg is converted by arginase I and arginase II to urea and ornithine and oxidized by the inducible form of nitric oxide synthase to citrulline and nitric oxide. Thus, L-Arg is profoundly reduced in cancer patients, following liver transplantation, or in severe trauma by an increased production of arginase I (Zea et al., 2005; Roth et al., 1994; Angele et al., 1999). Increased arginase activity is frequently observed in patients with breast, prostate, lung and colon cancer (Cederbaum et al., 2004). This results in a decreased T-cell proliferation and an impaired T-cell function. GCN2-knockout T cells did not show a decreased proliferation in the absence of L-Arg (Rodriguez et al., 2006).

GCN2 activation via L-Arg deprivation was also shown to occur in astrocytes, similar to how activation of GCN2 kinase mediates proliferative arrest and T-cell anergy induction in response to Trp deprivation by IDO. L-Arg consumption by arginase 1 in tumor-conditioned ges mediated GCN2 kinase-dependent cell cycle arrest in the G0-G1 phase and downregulation of the ζ chain of the TCR/CD3 complex in antigen-activated T cells (Rodriguez et al., 2002).

Activation of GCN2 through amino acid deficient-conditions promotes macrophage inflammation and mortality in a mouse model of septicemia. GCN2 knockout macrophages had a significant reduction of cytokine gene expression after lipopolysaccharide (LPS) stimulation. When monocytic-lineage-specific GCN2 knockout mice were challenged with a lethal dose of LPS, mice showed reduced inflammatory responses, with decreased IL-6 and IL-12 expression correlating with significant reduction in animal mortality (Liu et al., 2014).

In some embodiments, provided herein is a method of treating a disease mediated by the GCN2 pathway in an individual comprising administering an effective amount of a compound of Formula (I) and Formula (Ia), or a pharmaceutically acceptable salt thereof, to the individual.

In some embodiments, provided herein is a method of treating a disease characterised by activation of the GCN2 pathway in an individual comprising administering an effective amount of a compound of Formula (I) and Formula (Ia), or a pharmaceutically acceptable salt thereof, to the individual.

In some embodiments, provided herein is a method of treating a disease in an individual, wherein the individual has a low level of an amino acid. In some embodiments, the individual has a low level of a nonessential amino acid. In some embodiments, the individual has a low level of L-arginine. In some embodiments, the individual has a low level of L-tryptophan. In some embodiments, the disease results in a low level of L-arginine in a specific tissue or cell type, such as a tumor or an immune cell. In some embodiments, the disease results in a low level of L-tryptophan in a specific tissue or cell type, such as a tumor or an immune cell. In some embodiments, the level of L-tryptophan is less than 200 μM, less than 100 μM, less than 75 μM, less than 50 μM, or less than 25 μM. In some embodiments, the level of L-tryptophan is 10 μM to 75 μM. In some embodiments, the level of L-arginine is less than 200 μM, less than 100 μM, less than 75 μM, less than 50 μM, or less than 25 μM. In some embodiments, the level of L-arginine is 10 μM to 75 μM.

Also provided herein is a method of treating a disease in an individual, wherein the disease involves overexpression of GCN2. In some embodiments, provided herein is a method of treating a disease in an individual, wherein the disease involves activation of GCN2. In some embodiments, GCN2 is overexpressed and/or activated in a in specific tissue or cell type, such as a tumor or an immune cell.

In some embodiments, the methods provided herein inhibit a stress response in a cell. In some embodiments, the stress response is involved protecting cancer cells. In some embodiments, the stress response relates to amino acid starvation. In some embodiments, the stress response is a the unfolded protein response. In some embodiments, the stress response is an ER stress response.

In some embodiments, the methods provided herein result in reduced phosphorylation of GCN2. In some embodiments, downstream signalling by GCN2 is reduced. In some embodiments, phosphorylation of eIF2α kinase is reduced.

It was described that persistent parasite or viral infections are associated to the local induction of IDO expression affecting the activation of a proper immune response. It was also demonstrated that cutaneous Leishmania major infection stimulated expression of IDO in local lymph nodes. Induced IDO attenuated the T cell stimulatory functions of dendritic cells and suppressed local T cell responses to exogenous and nominal parasite antigens (Makala et al., 2011).

It was also demonstrated the role of IDO in leprosy. An increased number of macrophages/dendritic cells expressing IDO were found in lepromatous compared to reversal reaction patients. Furthermore, an increased IDO message in Mycobacterium leprae-stimulated peripheral blood mononuclear cells were also found. These data suggest that M. leprae chronic infection activates the suppressive molecule IDO which, in turn, contributes to the specific immunosuppression observed in lepromatous leprosy (de Souza et al., 2011).

It was described that HIV inhibits CD4+ T-cell proliferation by inducing IDO in plasmacytoid dendritic cells and that in vitro inhibition of IDO results in increased CD4(+) T-cell proliferative response in peripheral blood mononuclear cells from HIV-infected patients (Boasso et al., 2007).

Accordingly, inhibitors of the IDO/GCN2 pathway as disclosed herein could be used to enhance immune responses to chronic and persistent infections. In some embodiments, a compound or salt thereof described herein or a composition described herein may be used in a method of treating or preventing a viral infection. In some embodiments, the viral infection is an African swine fever virus, a dengue virus, an enterovirus, a hepatitis B virus, a hepatitis C virus, influenza virus, a tick-borne encephalitis virus, or a West Nile virus infection. In some embodiments, the viral infection is caused by a virus that activates GCN2 in an infected cell.

The basic mechanism of nutritional stress management mediated by GCN2 pathway functions primarily to couple cell growth to amino acid availability (Zhang et al., 2002).

In the tumor microenvironment, the abnormal development of vasculature results in insufficient blood supply and deprivation of glucose and amino acids. Both amino acid and glucose deprivation, stresses found in solid tumors, activated GCN2 to upregulate ATF4 target genes involved in amino acid synthesis and transport. GCN2 activation/overexpression and increased phospho-eIF2α were observed in human and mouse tumors compared with normal tissues and abrogation of ATF4 or GCN2 expression significantly inhibited tumor growth in vivo (Ye et al., 2010).

ATF4 is necessary for tumor cells to maintain homeostasis of amino acid metabolism and that activation of GCN2-ATF4-asparagine synthetase (ASNS) pathway promotes tumor cell survival under nutrient (amino acid or glucose) deprivation. GCN2-eIF2α pathway is activated in various human and mouse tumor tissues. Deficiency of ATF4 or GCN2 severely inhibits tumor growth in vivo. Together, these results suggest that GCN2-ATF4-ASNS pathway is a promising target for tumor therapy.

Tumor xenograft studies of head and neck squamous cell carcinoma (HNSCC), or fibrosarcoma (HT1080) cell lines with GCN2 deletions prevented tumor growth and survival (Ye et al., 2010; Wang et al., 2013). Additionally, in response to vemurafenib, BRAF-mutated melanoma and colorectal cancer cells rapidly induced the ISR as a cytoprotective mechanism through activation of GCN2. The vemurafenib-triggered ISR, an event independent of downstream MEK inhibition, was specifically prevented by silencing GCN2, but not other eIF2α kinases. Interestingly, ATF4 silencing by siRNA rendered BRAF-mutated melanoma cells sensitive to vemurafenib. Thus, the GCN2-mediated ISR can promote cellular adaptation to vemurafenib-induced stress, providing an insight into the development of drug resistance (Nagasawa et al., 2017).

It was reported that amino acid deficiency, glucose deprivation and hypoxia promote tumor growth and angiogenesis through the GCN2/eIF2α/ATF4 pathway (Wang et al., 2013). GCN2 expression is elevated in human tumors to overcome the stress associated to amino acid deprivation by stimulating vascular endothelial growth factor (VEGF)—mediated angiogenesis.

Leukemia cells lack the ability to synthesize asparagine. Thus, asparaginase, which functions by depletion of asparagine and glutamine, is a first line of treatment for B-Cell-derived acute lymphoblastic leukemia (B-ALL) (Terwilliger et al., 2017). It has been demonstrated that treatment with asparaginase activates GCN2 pathway in several leukemia cells and this is a mechanism by which tumor cells cope with nutrient stress by reversing chemotherapeutic amino acid deprivation (Lough et al., 2018). The inhibition of GCN2 sensitizes cancer cells with low basal-level expression of asparagine synthetase (ASNS) to the anti-leukemic agent asparaginase (Nakamura et al., 2018). Thus, GCN2 inhibitors could be exploited as a single agent therapy or in combination with asparaginase.

Without being bound to any particular theory, the GCN2-eIF2α-ATF4 pathway is important for maintaining metabolic homeostasis in tumor cells, making it a novel and attractive target for anti-tumor approaches.

Compounds as GCN2 modulators as disclosed herein can be useful as a prophylactic or therapeutic agent for many GCN2 associated diseases such as cancer: colorectal cancer, gastrointestinal stromal tumor, lung cancer (e.g. small and non-small cell lung cancer, malignant mesothelioma, primary lung cancer), hematologic cancer (e.g. multiple myeloma, leukemia (e.g. acute myeloid leukemia, acute lymphocytic leukemia, chronic leukemia), malignant lymphoma, Hodgkin's disease, non-Hodgkin's leukemia, chronic myeloproliferative disease), cancer metastasis, precancerous lesions (e.g. bone marrow myelodysplastic syndrome), pancreatic cancer (e.g. pancreatic duct cancer, pancreatic endocrine tumor), pharyngeal cancer, laryngeal cancer, esophagus cancer, gastric cancer (e.g. papillary adenocarcinoma, adenosquamous carcinoma), duodenal cancer, small intestinal cancer, breast cancer (e.g. ductal carcinoma in situ, inflammatory breast cancer, invasive ductal carcinoma), ovarian cancer (e.g. ovarian epithelial carcinoma, ovarian germ cell tumor), testis tumor, prostate cancer (e.g. hormone and non-hormone dependent prostate cancer, castration-resistant prostate cancer), liver cancer (e.g. hepatoma, primary liver cancer), extrahepatic bile duct cancer, thyroid cancer, renal cancer (e.g. renal cell carcinoma, clear cell renal carcinoma), uterine cancer (e.g. cervix cancer, uterine body cancer, uterus sarcoma), brain tumor (e.g. glioma, glioblastoma, medulloblastoma, astrocytoma, hypophyseal adenoma), retinoblastoma, skin cancer (e.g. melanoma, basal cell carcinoma), sarcoma (e.g. rhabdomyosarcoma, leiomyosarcoma, soft tissue sarcoma, osteosarcoma, spindle cell sarcoma), malignant bone tumor, urinary bladder cancer. In some embodiments, a compound or salt thereof described herein or a composition described herein may be used in a method of treating cancer, such as breast cancer, colorectal cancer, ovarian cancer, prostate cancer, pancreatic cancer, kidney cancer, lung cancer, melanoma, fibrosarcoma, bone sarcoma, connective tissue sarcoma, renal cell carcinoma, giant cell carcinoma, squamous cell carcinoma, leukemia, skin cancer, soft tissue cancer, liver cancer, gastrointestinal carcinoma, or adenocarcinoma, hepatocellular carcinoma, thyroid cancer, multiple myeloma, cancer of secretory cells, myelodysplastic syndrome, myeloproliferative neoplasm, malignant glioma, non-Hodgkin's lymphoma, Hodgkin's lymphoma, Burkitt's lymphoma, chronic lymphocytic leukemia, chronic myeloid leukemia, hairy cell leukemia, monoclonal gammopathy of undetermined significance (MGUS), plasmacytoma, lymphoplasmacytic lymphoma, acute lymphoblastic leukemia, acute myeloid leukemia, chronic myelomonocytic leukemia, juvenile myelomonocytic leukemia, large granular lymphocytic leukemia, B-cell prolymphocytic leukemia, T-cell prolymphocytic leukemia. small cell lung cancer, malignant pleural mesothelioma, Head and neck squamous cell carcinoma, glioblastoma multiforme, sarcoma or pediatric neuroblastoma. In some embodiments, the compound, salt, or composition may be used in a method of treating metastatic kidney cancer, chronic lymphocytary leukemia, pancreatic adenocarcinoma, or non-small cell lung cancer.

The GCN2 modulation provides the opportunity for interfering with the tumor growth metabolism at the same time what may enhance the efficacy of a monotherapy or a combination therapy with other anticancer agents. In some embodiments, a compound or salt thereof described herein or a composition described herein may be used in treating tumor in combination with other anticancer agents such as an anti-neoplastic agent, an immune checkpoint inhibitor, or any other suitable anti-cancer agent. Exemplary immune checkpoint inhibitors include anti-PD-1, anti-PD-L1, anti GITR, anti-OX-40, anti-LAG3, anti-TIM-3, anti-41BB, anti-CTLA-4 antibodies. Exemplary anti-neoplastic agents can include, for example, anti-microtubule agents, platinum coordination complexes, alkylating agents, topoisomerase II inhibitors, topoisomerase I inhibitors, antimetabolites, antibiotic agents, hormones and hormonal analogs, signal transduction pathway inhibitors, non-receptor tyrosine kinase angiogenesis inhibitors, proteasome inhibitors, and inhibitors of cancer metabolism. Other anti-cancer agents can include one or more of an immuno-stimulant, an antibody or fragment thereof (e.g. an anti-CD20, anti-HER2, anti-CD52, or anti-VEGF antibody or fragment thereof), or an immunotoxin (e.g. an anti-CD33 antibody or fragment thereof, an anti-CD22 antibody or fragment thereof, a calicheamicin conjugate, or a pseudomonas exotoxin conjugate).

In some embodiments, a compound or salt thereof described herein or a composition described herein may be used in a method of treating cancer in an individual, wherein one or more cancer cells in the individual are dormant cancer cells. In some embodiments, one or more of the dormant cancer cells are disseminated tumor cells or circulating tumor cells. In some embodiments, one or more of the dormant cancer cells are disseminated tumor cells.

In some embodiments, a compound or salt thereof described herein or a composition described herein may be used in a method of treating cancer in an individual, wherein the individual has had a prior treatment. In some embodiments, the cancer is resistant or refractory to the prior treatment. In some embodiments, the cancer has progressed on the prior treatment. In the embodiments, the cancer is a recurrent cancer. In some embodiments, the prior treatment was treatment with a ubiquitin-proteasome pathway inhibitor (e.g., bortezomib), a taxane (e.g., paclitaxel or docetaxel), a Cox-2 inhibitor (e.g., celecoxib), a platinum-based antineoplastic drug (e.g., cisplatin or oxaliplatin), an anthracycline (e.g. doxorubicin), a pyrimidine analog (e.g. 5-fluorouracil or gemcitabine), a topoisomerase inhibitor (e.g., etoposide), an mTOR inhibitor (e.g., rapamycin), an immune-check point inhibitor, or an agent that is used in immune oncology. In some embodiments, the cancer is resistant to treatment with a ubiquitin-proteasome pathway inhibitor (e.g., bortezomib), a taxane (e.g., paclitaxel or docetaxel), a Cox-2 inhibitor (e.g., celecoxib), a platinum-based antineoplastic drug (e.g., cisplatin or oxaliplatin), an anthracycline (e.g. doxorubicin), a pyrimidine analog (e.g. 5-fluorouracil or gemcitabine), a topoisomerase inhibitor (e.g., etoposide), an mTOR inhibitor (e.g., rapamycin), an immune-check point inhibitor, or an agent that is used in immune oncology. In some embodiments, the cancer is resistant to treatment with doxorubicin and/or rapamycin.

In some embodiments, the administration of the compound, salt, or composition reduces tumor growth, tumor proliferation, or tumorigenicity in the individual. In some embodiments, the compound, salt, or composition may be used in a method of reducing tumor growth, tumor proliferation, or tumorigenicity in an individual in need thereof. In some embodiments, tumor growth is slowed or arrested. In some embodiments, tumor growth is reduced at least about 10%, 20%, 30%, 40%, 50%, 60%, 70%, 80%, or 90%. In some embodiments, the tumor is reduced in size. In some embodiments, tumor size is reduced at least about 10%, 20%, 30%, 40%, 50%, 60%, 70%, 80%, or 90%. In some embodiments, tumor metastasis is prevented or slowed. In some embodiments, the tumor growth, tumor proliferation, or tumorigenicity is compared to the tumor growth, tumor proliferation, or tumorigenicity in the individual prior to the administration of the compound, salt, or composition. In some embodiments, the tumor growth, tumor proliferation, or tumorigenicity is compared to the tumor growth, tumor proliferation, or tumorigenicity in a similar individual or group of individuals. Methods of measuring tumor growth, tumor proliferation, and tumorigenicity are known in the art, for example by repeated imaging of the individual.

In some embodiments, the administration of the compound, salt, or composition induces apoptosis of cancer cells. In some embodiments, apoptosis of cancer cells is increased at least 10%, at least 20%, at least 30%, at least 40% or at least 50% upon administration.

In some embodiments, the administration of the compound, salt, or composition reduces CHOP induction. In some embodiments, CHOP induction is reduced at least 10%, at least 20%, at least 30%, at least 40% or at least 50% upon administration.

In some embodiments, the administration of the compound, salt, or composition does not induce PERK activation. In some embodiments, CHOP production caused by PERK is not inhibited upon administration.

It was recently demonstrated that hepatic GCN2 is activated in fatty livers, and GCN2 deficiency protects against high-fat diet (HFD)-induced hepatic steatosis and insulin resistance, indicating that the role of GCN2 in lipid metabolism regulation is context dependent.

After high fat diet (HFD) feeding for 12 weeks, Gcn2−/− mice were less obese than wild-type (WT) mice, and Gcn2−/− significantly attenuated HFD-induced liver dysfunction, hepatic steatosis and insulin resistance. In the livers of the HFD-fed mice, GCN2 deficiency resulted in higher levels of lipolysis genes, lower expression of genes related to fatty acid synthesis, transport and lipogenesis, and less induction of oxidative stress. In this work it was also reported that knockdown of GCN2 attenuated, whereas overexpression of GCN2 exacerbated, palmitic acid-induced steatosis, oxidative & ER stress, and changes of peroxisome proliferator-activated receptor gamma (PPARγ), fatty acid synthase and metallothionein expression in HepG2 cells (Liu et al., 2018). These findings suggest that strategies to inhibit GCN2 activity in the liver may provide a novel approach to attenuate Nonalcoholic fatty liver disease (NAFLD) development.

When mice are reared with a medium fat (22 kcal % fat) diet during perinatal development, GCN2 deficiency reduced hepatic triglyceride storage (Xu et al., 2013).

It was reported that GCN2 deficiency attenuated cardiac dysfunction and hyperlipidemia in both type 1 diabetes (T1D) and Type 2 diabetes (T2D) mice, and the improved cardiac function in diabetic GCN2−/− mice was associated with reduced hypertrophy, fibrosis, lipid accumulation, oxidative stress, inflammation and apoptosis. The pathological role of GCN2 in diabetic cardiomyopathy (DCM) was also validated in high glucose or palmitic acid treated H9C2 rat cardio-myoblast cell line (Feng et al., 2019).

GCN2 deficiency attenuated transverse aortic constriction (TAC)-induced cardiac dysfunction and cardiomyocyte apoptosis by decreasing cardiomyocyte apoptosis and myocardial oxidative stress. GCN2 activation impairs adaptative responses to congestive heart failure (Lu et al., 2014). After doxorubicin-induced cardiac dysfunction, Gcn2−/− mice developed less contractile dysfunction, myocardial fibrosis, apoptosis, and oxidative stress compared with WT mice. In the hearts of the Dox-treated mice, GCN2 deficiency attenuated eIF2α phosphorylation and induction of its downstream targets, activating transcription factor 4 (ATF4) and C/EBP homologous protein (CHOP), and preserved the expression of anti-apoptotic factor Bcl-2 and mitochondrial uncoupling protein-2(UCP2) (Wang et al., 2018). These data suggest that strategies to inhibit GCN2 activity in cardiomyocyte may provide a novel approach to attenuate Dox-related cardiotoxicity.

Accordingly, in some embodiments, a compound or salt thereof described herein or a composition described herein may be used in treating or preventing metabolic and cardiac diseases.

It was demonstrated that GCN2 deletion attenuates denervation-induced muscle atrophy. GCN2 deficiency also significantly attenuated the muscle mass loss in atrophied gastrocnemius and extensor digitorum longus muscles. Similar results were observed on day 14 after denervation. Wheat germ agglutinin staining of muscle cryosections demonstrated that GCN2-deficient TA muscles had a better preservation of myofiber size in response to denervation (Guo et al., 2018). Furthermore, the detrimental effect of GCN2 in denervation-induced muscle atrophy was related to FoxO3a activation, which upregulates genes involved in both the ubiquitin-proteasome pathway and autophagy in muscle atrophy (Sandri et al., 2004; Bertaggia et al., 2012; Wei et al., 2013; Guo et al., 2016).

Accordingly, in some embodiments, a compound or salt thereof described herein or a composition described herein may be used in treating or preventing muscle atrophy.

Expression of long-lasting synaptic plasticity and long-term memory (LTM) requires protein synthesis, which can be repressed by phosphorylation of eIF2α. In mice lacking the eIF2α kinase, GCN2, the reduction in phosphorylated eIF2α is associated with altered synaptic plasticity and memory. GCN2 deficient mice, in which both eIF2α phosphorylation and ATF4 levels are reduced, the threshold for long-lasting long-term potentiation (L-LTP) and LTM in hippocampus is lowered and it is associated with an improved spatial memory of weak conditioning (Costa-Mattioli et al., 2005). This model is supported by the increase of ATF4 expression upon treatment with an inhibitor of eIF2α phosphatases, Sal003, which leads to an impairment of L-LTP and LTM.

Accordingly, in some embodiments, a compound or salt thereof described herein or a composition described herein may be used in treating or preventing memory loss.

Elevated phosphorylation of eIF2α has been observed in the brains of Alzheimer's disease (AD) patients and Alzheimer's disease model mice. Suppressing GCN2 prevents amyloid-β-induced synaptic plasticity impairments by diminishing eIF2α phosphorylation. Senile plaques are primarily composed of β-amyloid peptides (Aβ) derived from amyloid precursor protein (APP) that has undergone proteolytic processing by β-secretase (BACE-1) and γ-secretase. It was also reported that BACE-1 levels are translationally increased by phosphorylation of elF2α (O'Connor et al., 2008). Inhibition of GCN2 under such disease conditions that promote activation of γ-secretase or induction of BACE-1 with consequence of accumulation of Aβ and plaque formation in the brain would provide a valuable avenue to cope with or even stop the progression of neurodegenerative diseases.

Deletion of GCN2 prevented impairments of synaptic plasticity and defects in spatial memory in mice that express familial AD—related mutations in amyloid precursor protein (APP) and presenilin-1 (PS1). PS1 is essential for γ-secretase activity and GCN2/eIF2α/ATF4 signalling has an important role in the regulation of γ-secretase activity in autophagy impaired cells (Ohata et al., 2010).

Furthermore, hippocampal LTP deficits in APP-PS1 mice were normalized in APP-PS1 GCN2-deficient mice. The impairments in spatial learning and memory displayed by the APP-PS1 mice were prevented in the APP-PS1 GCN2-deficient mice as observed in a Morris Water Maze task by decreased escape latency, increased platform crossings as well as target quadrant occupancy that were close to those displayed by wild-type mice (Ma et al., 2013). Taken together, these findings indicate that genetic removal of the eIF2α kinase GCN2 prevents Alzheimer's disease-associated LTP failure and spatial memory impairments.

Accordingly, in some embodiment, a compound or salt thereof described herein or a composition described herein may be used in treating neurodegenerative diseases.

Angiogenesis, the formation of new blood vessels by endothelial cells (ECs), is an adaptive response to oxygen/nutrient deprivation sensed by GCN2 and orchestrated by vascular endothelial growth factor (VEGF) upon ischemia or exercise. Neovascularization in the retina and the choroid is a major cause of vision loss in severe eye diseases, such as diabetic retinopathy, age-related macular degeneration, retinopathy of prematurity, and central and branch retinal vein occlusion. Amino acid restriction triggers angiogenesis via GCN2/ATF4 regulation of VEGF and hydrogen sulfide production (Longchamp et al., 2018).

Yet, retinal neovascularization is causally and dynamically associated with vasodegeneration, ischemia, and vascular remodeling in retinal tissues (Zhang et al., 2015). ATF4 has been shown to function as an oxygen sensor and interacts with HIF-1α to regulate VEGF production (Zhong et al., 2012). Global knockout of ATF4 disturbs lens development, resulting in microphthalmia (Masuoka and Townes, 2002). Genetic inhibition of ATF4 activity attenuates diabetic-induced retinal inflammation and vascular leakage, suggesting that up-regulation of ATF4 contributes to retinal inflammation and endothelial barrier dysfunction in diabetic retinopathy (Chen et al., 2012).

Accordingly, in some embodiment, a compound or salt thereof described herein or a composition described herein may be used in treating ocular diseases.

Since there is accumulating evidence showing that the GCN2 pathway strongly influences the function of the immune system, the present invention encompasses the use of GCN2 modulators for the treatment or prevention of immune related disorders. In some embodiment, a compound or salt thereof described herein or a composition described herein may be used in treating or preventing immune related disorders. In some embodiment, a compound or salt thereof described herein or a composition described herein may be used in treating or preventing an autoimmune disease selected from the group consisting of arthritis, graft-versus-host disease, Crohn's disease, multiple sclerosis, lupus, type 1 diabetes mellitus, rheumatoid arthritis, Grave's disease, autoimmune hemolytic anemia, Wegener's granulomatosis, ankylosing spondylitis, aplastic anemia, Behcet's disease, hyper IgE syndrome, idiopathic thrombocytopenia purpura, Myasthenia gravis and psoriasis.

In some embodiment, a compound or salt thereof described herein or a composition described herein may be used in transplantation proceeding to treat organ rejection, myeloablative and non-myeloablative bone marrow transplant rejection.

In some embodiment, a compound or salt thereof described herein or a composition described herein may be used to inhibit the phosphorylation of GCN2. In some embodiments, a compound or salt thereof described herein or a composition described herein can be used to promote the recovery protein synthesis under amino acid deprived condition. In some embodiments, a compound or salt thereof described herein or a composition described herein can be used to enhance protein synthesis, and thus can be used in diseases or disorders that are mediated by a decrease in protein synthesis such as muscle atrophy, muscle dystrophy, cachexia, synaptic plasticity and long-term memory, among others.

In accordance with the present disclosure, in some embodiments, the individual is a mammal. In some embodiments, the individual is a primate, bovine, ovine, porcine, equine, canine, feline, rabbit, or rodent. In some embodiments, the individual is a human. In some embodiments, the individual has any of the diseases or disorders disclosed herein. In some embodiments, the individual is a risk for developing any of the diseases or disorders disclosed herein.

Also provided herein are uses of a compound described herein or a pharmaceutically acceptable salt thereof, or a pharmaceutical composition described herein, in the manufacture of a medicament. In some embodiments, the manufacture of a medicament is for the treatment of a disorder or disease described herein. In some embodiments, the manufacture of a medicament is for the prevention and/or treatment of a disorder or disease mediated by the GCN2 pathway.

In some embodiments, a compound or salt thereof described herein or a composition described herein may be used in a method as either a stand-alone therapy, or as a conjunctive therapy with other agents that are either palliative (e.g., agents that relieve the symptoms of the disorder to be treated), and/or agents that target the etiology of the disorder. Compounds or compositions of the present invention may be used or administered in combination with a second therapeutic agent. Compounds or compositions of the present invention may be used or administered in combination with an anticancer agent, anti-angiogenesis agent or an agent that targets an immune checkpoint protein. Compounds or compositions of the present invention may be used or administered in combination with a second therapeutic agent selected from: PEG-arginase, asparaginase, anti-angiogenic factors, cysteinase or sulfasalazine.

As provided herein, compounds or salts thereof described herein and compositions described herein may be administered with an agent to treat any of the diseases and disorders disclosed herein. In some embodiments, the agent is an anti-angiogenesis agent. In some embodiments, the agent is an anticancer agent. In some embodiments, the agent targets an immune checkpoint protein.

In some embodiments, (a) a compound described herein, or a pharmaceutically acceptable salt thereof, or a pharmaceutical composition described herein and (b) an agent are sequentially administered, concurrently administered or simultaneously administered. In certain embodiments, (a) a compound described herein, or a pharmaceutically acceptable salt thereof, or a pharmaceutical composition described herein and (b) an agent are administered with a time separation of about 15 minutes or less, such as about any of 10, 5, or 1 minutes or less. In certain embodiments, (a) a compound described herein, or a pharmaceutically acceptable salt thereof, or a pharmaceutical composition described herein and (b) an agent are administered with a time separation of about 15 minutes or more, such as about any of 20, 30, 40, 50, 60, or more minutes. Either (a) a compound described herein, or a pharmaceutically acceptable salt thereof, or a pharmaceutical composition described herein and (b) an agent may be administered first. In certain embodiments, (a) a compound described herein, or a pharmaceutically acceptable salt thereof, or a pharmaceutical composition described herein and (b) an agent are administered simultaneously.

Provided herein is a method of enhancing an immune response in an individual comprising administering to the individual (a) a compound described herein, or a pharmaceutically acceptable salt thereof, or a pharmaceutical composition described herein and (b) an agent that targets an immune checkpoint protein. In some embodiments, the individual has cancer. In some embodiments, the enhanced immune response is directed to a tumor or cancerous cell.

Also provided herein are methods of treating cancer in an individual in need thereof comprising administering to the individual (a) a compound described herein, or a pharmaceutically acceptable salt thereof, or a pharmaceutical composition described herein and (b) an agent that targets an immune checkpoint protein, wherein an immune response of the individual is increased.

In some embodiments, the agent is an anticancer agent. In some embodiments, anticancer agent is an ubiquitin-proteasome pathway inhibitor (e.g., bortezomib), a taxane (e.g., paclitaxel or docetaxil), a Cox-2 inhibitor (e.g., celecoxib), a platinum-based antineoplastic drug (e.g., cisplatin or oxaliplatin), an anthracycline (e.g. doxorubicin), a pyrimidine analog (e.g. 5-fluorouracil or gemcitabine), a topoisomerase inhibitor (e.g., etoposide), or an agent that modulates the Unfolded Protein Response or the Integrated Stress Response (e.g. an IRE1/XBP1 inhibitor or a PERK inhibitor). In some embodiments, the anticancer agent is oxaliplatin, 5-fluorouracil, or gemcitabine. In some embodiments, the anticancer agent is an immune-check point inhibitor, or an agent that is used in immune oncology.

In some embodiments, an effective amount of a compound described herein, or a pharmaceutically acceptable salt thereof, or a pharmaceutical composition described herein is administered to an individual with cancer to increase sensitivity to one or more anticancer treatments.

In some embodiments, an effective amount of a compound described herein, or a pharmaceutically acceptable salt thereof, or a pharmaceutical composition described herein is administered to an individual with cancer to increase sensitivity to radiation. In some embodiments, provided herein are methods of treating cancer in an individual in need thereof comprising administering to the individual (a) a compound described herein, or a pharmaceutically acceptable salt thereof, or a pharmaceutical composition described herein and (b) radiation.

In some embodiments, an effective amount of a compound described herein, or a pharmaceutically acceptable salt thereof, or a pharmaceutical composition described herein is administered to an individual with cancer to increase sensitivity to one or more anticancer agents. In some embodiments, the anticancer agent is an ubiquitin-proteasome pathway inhibitor (e.g., bortezomib), a taxane (e.g., paclitaxel or docetaxil), a Cox-2 inhibitor (e.g., celecoxib), a platinum-based antineoplastic drug (e.g., cisplatin or oxaliplatin), an anthracycline (e.g. doxorubicin), a pyrimidine analog (e.g. 5-fluorouracil or gemcitabine), a topoisomerase inhibitor (e.g., etoposide), or an agent that modulates the Unfolded Protein Response or the Integrated Stress Response (e.g. an IRE1/XBP1 inhibitor or a PERK inhibitor). In some embodiments, the anticancer agent is oxaliplatin, 5-fluorouracil, or gemcitabine. In some embodiments, the anticancer agent is an immune-check point inhibitor, or an agent that is used in immune oncology.

### Definitions

In this application, the following definitions apply, unless indicated otherwise.

The term “GCN2 modulator” as used herein refers to any compound which binds to and modulates the function of GCN2. The term “modulator” should be interpreted to include modulation by modalities including, but not limited to, antagonists, agonists, partial agonists and inverse agonists.

The term “treatment”, in relation to the uses of any of the compounds described herein, including those of Formula (1) is used to describe any form of intervention where a compound is administered to a subject suffering from, or at risk of suffering from, or potentially at risk of suffering from the disease or disorder in question, such as cancer or an immunological disease. Thus, the term “treatment” covers both preventative (prophylactic) treatment and treatment where measurable or detectable symptoms of the disease or disorder are being displayed. Treatment also encompasses decreasing one or more symptoms resulting from the disease or disorder, diminishing the extent of the disease or disorder, stabilizing the disease or disorder (e.g., preventing or delaying the worsening of the disease or disorder), delaying the occurrence or recurrence of the disease or disorder, delaying or slowing the progression of the disease or disorder, ameliorating the disease or disorder state, providing a remission (whether partial or total) of the disease or disorder, decreasing the dose of one or more other medications required to treat the disease or disorder, enhancing the effect of another medication used to treat the disease or disorder, delaying the progression of the disease or disorder, increasing the quality of life, and/or prolonging survival of a patient.

In some variations, treatment does not include prevention. Thus, it is understood that in some variations, treatment refers to uses of any of the compounds described herein, including those of Formula (1) is used to describe any form of intervention where a compound is administered to a subject suffering from, or at risk of suffering from, or potentially at risk of suffering from the disease or disorder in question, such as cancer or an immunological disease.

“Individual” refers to mammals and includes humans and non-human mammals. Examples of individuals include, but are not limited to mice, rats, hamsters, guinea pigs, pigs, rabbits, cats, dogs, goats, sheep, cows, and humans. In some embodiments, individual refers to a human.

The term “effective therapeutic amount” (for example in relation to methods of treatment of a disease or condition) refers to an amount of the compound which is effective to produce a desired therapeutic effect. For example, if the condition is pain, then the effective therapeutic amount is an amount sufficient to provide a desired level of pain relief. The desired level of pain relief may be, for example, complete removal of the pain or a reduction in the severity of the pain. With respect to the treatment, if the condition is cancer, then the effective therapeutic amount is an amount sufficient to reduce one or more symptoms associated with cancer, for example reduction of tumour size or rate of metastasis.

As used herein, “about” a parameter or value includes and describes that parameter or value per se. For example, “about X” includes and describes X per se.

Terms such as “alkyl”, “alkoxy” and “halo” are all used in their conventional sense (e.g. as defined in the IUPAC Gold Book), unless indicated otherwise. “Optionally substituted” as applied to any group means that the said group may if desired be substituted with one or more substituents, which may be the same or different.

“Heteroaryl” as used herein refers to an unsaturated aromatic cyclic group having from 1 to 14 annular carbon atoms and at least one annular heteroatom, including but not limited to heteroatoms such as nitrogen, oxygen, and sulfur. A heteroaryl group may have a single ring (e.g., pyridyl, furyl) or multiple condensed rings (e.g., indolizinyl, benzothienyl) which condensed rings may or may not be aromatic. Particular heteroaryl groups are 5 to 14-membered rings having 1 to 12 annular carbon atoms and 1 to 6 annular heteroatoms independently selected from nitrogen, oxygen, and sulfur, 5 to 10-membered rings having 1 to 8 annular carbon atoms and 1 to 4 annular heteroatoms independently selected from nitrogen, oxygen, and sulfur, or 5, 6 or 7-membered rings having 1 to 5 annular carbon atoms and 1 to 4 annular heteroatoms independently selected from nitrogen, oxygen, and sulfur. In one variation, particular heteroaryl groups are monocyclic aromatic 5-, 6- or 7-membered rings having from 1 to 6 annular carbon atoms and 1 to 4 annular heteroatoms independently selected from nitrogen, oxygen and sulfur. In another variation, particular heteroaryl groups are polycyclic aromatic rings having from 1 to 12 annular carbon atoms and 1 to 6 annular heteroatoms independently selected from nitrogen, oxygen, and sulfur. A heteroaryl group may be fused with aryl, cycloalkyl, or heterocyclyl. In one variation, a heteroaryl group having more than one ring where at least one ring is aryl, cycloalkyl, or heterocyclyl is connected to the parent structure at an atom in the aromatic cyclic group having at least one annular heteroatom. A heteroaryl group may be connected to the parent structure at a ring carbon atom or a ring heteroatom.

“Heterocycle”, “heterocyclic”, or “heterocyclyl” as used herein refers to a saturated or an unsaturated non-aromatic cyclic group having a single ring or multiple condensed rings, and having from 1 to 14 annular carbon atoms and from 1 to 6 annular heteroatoms, such as nitrogen, sulfur or oxygen, and the like. In certain embodiments, the nitrogen and/or sulfur atom(s) of the heterocyclic group are optionally oxidized to provide for N-oxide, —S(O)—, or —SO₂— moieties. A heterocycle comprising more than one ring may be fused, bridged or spiro, or any combination thereof, but excludes heteroaryl. The heterocyclyl group may be optionally substituted independently with one or more substituents described herein. Particular heterocyclyl groups are 3 to 14-membered rings having 1 to 13 annular carbon atoms and 1 to 6 annular heteroatoms independently selected from nitrogen, oxygen and sulfur, 3 to 12-membered rings having 1 to 11 annular carbon atoms and 1 to 6 annular heteroatoms independently selected from nitrogen, oxygen and sulfur, 3 to 10-membered rings having 1 to 9 annular carbon atoms and 1 to 4 annular heteroatoms independently selected from nitrogen, oxygen and sulfur, 3 to 8-membered rings having 1 to 7 annular carbon atoms and 1 to 4 annular heteroatoms independently selected from nitrogen, oxygen and sulfur, or 3 to 6-membered rings having 1 to 5 annular carbon atoms and 1 to 4 annular heteroatoms independently selected from nitrogen, oxygen and sulfur. In one variation, heterocyclyl includes monocyclic 3-, 4-, 5-, 6- or 7-membered rings having from 1 to 2, 1 to 3, 1 to 4, 1 to 5, or 1 to 6 annular carbon atoms and 1 to 2, 1 to 3, or 1 to 4 annular heteroatoms independently selected from nitrogen, oxygen and sulfur. In another variation, heterocyclyl includes polycyclic non-aromatic rings having from 1 to 12 annular carbon atoms and 1 to 6 annular heteroatoms independently selected from nitrogen, oxygen and sulfur. A heterocyclyl group may be fused with aryl, cycloalkyl, or heteroaryl. In one variation, a heterocyclyl group having more than one ring where at least one ring is aryl, cycloalkyl, or heteroaryl is connected to the parent structure at an atom in the non-aromatic cyclic group having at least one heteroatom.

To the extent that any of the compounds described have chiral centres, the present invention extends to all optical isomers of such compounds, whether in the form of racemates or resolved enantiomers. Also provided herein are, where applicable, any and all stereoisomers of the compounds depicted herein, including geometric isomers (e.g., cis/trans isomers or E/Z isomers), enantiomers, diastereomers, or mixtures thereof in any ratio, including racemic mixtures.

The invention described herein relates to all crystal forms, solvates and hydrates of any of the disclosed compounds however so prepared. To the extent that any of the compounds disclosed herein have acid or basic centres such as carboxylates or amino groups, then all salt forms of said compounds are included herein. In the case of pharmaceutical uses, the salt should be seen as being a pharmaceutically acceptable salt.

Salts or pharmaceutically acceptable salts that may be mentioned include acid addition salts and base addition salts. Such salts may be formed by conventional means, for example by reaction of a free acid or a free base form of a compound with one or more equivalents of an appropriate acid or base, optionally in a solvent, or in a medium in which the salt is insoluble, followed by removal of said solvent, or said medium, using standard techniques (e.g. in vacuo, by freeze-drying or by filtration). Salts may also be prepared by exchanging a counter-ion of a compound in the form of a salt with another counter-ion, for example using a suitable ion exchange resin.

Examples of pharmaceutically acceptable salts include acid addition salts derived from mineral acids and organic acids, and salts derived from metals such as sodium, magnesium, potassium and calcium.

Examples of acid addition salts include acid addition salts formed with acetic, 2,2-dichloroacetic, adipic, alginic, aryl sulfonic acids (e.g. benzenesulfonic, naphthalene-2-sulfonic, naphthalene-1,5-disulfonic and p-toluenesulfonic), ascorbic (e.g. L-ascorbic), L-aspartic, benzoic, 4-acetamidobenzoic, butanoic, (+) camphoric, camphor-sulfonic, (+)-(1S)-camphor-10-sulfonic, capric, caproic, caprylic, cinnamic, citric, cyclamic, dodecylsulfuric, ethane-1,2-disulfonic, ethanesulfonic, 2-hydroxyethanesulfonic, formic, fumaric, galactaric, gentisic, glucoheptonic, gluconic (e.g. D-gluconic), glucuronic (e.g. D-glucuronic), glutamic (e.g. L-glutamic), α-oxoglutaric, glycolic, hippuric, hydrobromic, hydrochloric, hydriodic, isethionic, lactic (e.g. (+)-L-lactic and (±)-DL-lactic), lactobionic, maleic, malic (e.g. (−)-L-malic), malonic, (±)-DL-mandelic, metaphosphoric, methanesulfonic, 1-hydroxy-2-naphthoic, nicotinic, nitric, oleic, orotic, oxalic, palmitic, pamoic, phosphoric, propionic, L-pyroglutamic, salicylic, 4-amino-salicylic, sebacic, stearic, succinic, sulfuric, tannic, tartaric (e.g. (+)-L-tartaric), thiocyanic, undecylenic and valeric acids.

Also encompassed are any solvates of the compounds and their salts. Preferred solvates are solvates formed by the incorporation into the solid state structure (e.g. crystal structure) of the compounds of the invention of molecules of a non-toxic pharmaceutically acceptable solvent (referred to below as the solvating solvent). Examples of such solvents include water, alcohols (such as ethanol, isopropanol and butanol) and dimethylsulfoxide. Solvates can be prepared by recrystallising the compounds of the invention with a solvent or mixture of solvents containing the solvating solvent. Whether or not a solvate has been formed in any given instance can be determined by subjecting crystals of the compound to analysis using well known and standard techniques such as thermogravimetric analysis (TGA), differential scanning calorimetry (DSC) and X-ray crystallography.

The solvates can be stoichiometric or non-stoichiometric solvates. Particular solvates may be hydrates, and examples of hydrates include hemihydrates, monohydrates and dihydrates. For a more detailed discussion of solvates and the methods used to make and characterise them, see Bryn et al, Solid-State Chemistry of Drugs, Second Edition, published by SSCI, Inc of West Lafayette, IN, USA, 1999, ISBN 0-967-06710-3.

The term “pharmaceutical composition” in the context of this invention means a composition comprising an active agent and comprising additionally one or more pharmaceutically acceptable carriers that is suitable for administration to an individual. The composition may further contain ingredients selected from, for example, diluents, adjuvants, excipients, vehicles, preserving agents, fillers, disintegrating agents, wetting agents, emulsifying agents, suspending agents, sweetening agents, flavouring agents, perfuming agents, antibacterial agents, antifungal agents, lubricating agents and dispersing agents, depending on the nature of the mode of administration and dosage forms. The compositions may take the form, for example, of tablets, dragees, powders, elixirs, syrups, liquid preparations including suspensions, sprays, inhalants, tablets, lozenges, emulsions, solutions, cachets, granules, capsules and suppositories, as well as liquid preparations for injections, including liposome preparations.

The compounds of the invention may contain one or more isotopic substitutions, and a reference to a particular element includes within its scope all isotopes of the element. For example, a reference to hydrogen includes within its scope ¹H, ²H (D), and ³H (T). Similarly, references to carbon and oxygen include within their scope respectively ¹²C, ¹³C and ¹⁴C and ¹⁶O and ¹⁸O. In an analogous manner, a reference to a particular functional group also includes within its scope isotopic variations, unless the context indicates otherwise. For example, a reference to an alkyl group such as an ethyl group or an alkoxy group such as a methoxy group also covers variations in which one or more of the hydrogen atoms in the group is in the form of a deuterium or tritium isotope, e.g. as in an ethyl group in which all five hydrogen atoms are in the deuterium isotopic form (a perdeuteroethyl group) or a methoxy group in which all three hydrogen atoms are in the deuterium isotopic form (a trideuteromethoxy group). The isotopes may be radioactive or non-radioactive.

Therapeutic dosages may be varied depending upon the requirements of the patient, the severity of the condition being treated, and the compound being employed. Determination of the proper dosage for a particular situation is within the skill of the art. Generally, treatment is initiated with the smaller dosages which are less than the optimum dose of the compound. Thereafter the dosage is increased by small increments until the optimum effect under the circumstances is reached. For convenience, the total daily dosage may be divided and administered in portions during the day if desired.

The magnitude of an effective dose of a compound will, of course, vary with the nature of the severity of the condition to be treated and with the particular compound and its route of administration. The selection of appropriate dosages is within the ability of one of ordinary skill in this art, without undue burden. In general, the daily dose range may be from about 10 g to about 30 mg per kg body weight of a human and non-human animal, preferably from about 50 g to about 30 mg per kg of body weight of a human and non-human animal, for example from about 50 g to about 10 mg per kg of body weight of a human and non-human animal, for example from about 100 g to about 30 mg per kg of body weight of a human and non-human animal, for example from about 100 g to about 10 mg per kg of body weight of a human and non-human animal and most preferably from about 100 g to about 1 mg per kg of body weight of a human and non-human animal.

### Pharmaceutical Formulations

While it is possible for the active compound to be administered alone, it is preferable to present it as a pharmaceutical composition (e.g., formulation).

Accordingly, in some embodiments of the invention, there is provided a pharmaceutical composition comprising at least one compound of Formula (1) as defined above together with at least one pharmaceutically acceptable excipient.

The pharmaceutically acceptable excipient(s) can be selected from, for example, carriers (e.g. a solid, liquid or semi-solid carrier), adjuvants, diluents (e.g solid diluents such as fillers or bulking agents; and liquid diluents such as solvents and co-solvents), granulating agents, binders, flow aids, coating agents, release-controlling agents (e.g. release retarding or delaying polymers or waxes), binding agents, disintegrants, buffering agents, lubricants, preservatives, anti-fungal and antibacterial agents, antioxidants, buffering agents, tonicity-adjusting agents, thickening agents, flavouring agents, sweeteners, pigments, plasticizers, taste masking agents, stabilisers or any other excipients conventionally used in pharmaceutical compositions.

The term “pharmaceutically acceptable” as used herein means compounds, materials, compositions, and/or dosage forms which are, within the scope of sound medical judgment, suitable for use in contact with the tissues of a subject (e.g. a human subject) without excessive toxicity, irritation, allergic response, or other problem or complication, commensurate with a reasonable benefit/risk ratio. Each excipient must also be “acceptable” in the sense of being compatible with the other ingredients of the formulation.

Pharmaceutical compositions containing compounds of the Formula (1) can be formulated in accordance with known techniques, see for example, Remington's Pharmaceutical Sciences, Mack Publishing Company, Easton, PA, USA. The pharmaceutical compositions can be in any form suitable for oral, parenteral, intravenous, intramuscular, intrathecal, subcutaneous, topical, intranasal, intrabronchial, sublingual, buccal, ophthalmic, otic, rectal, intra-vaginal, or transdermal administration.

Pharmaceutical dosage forms suitable for oral administration include tablets (coated or uncoated), capsules (hard or soft shell), caplets, pills, lozenges, syrups, solutions, powders, granules, elixirs and suspensions, sublingual tablets, wafers or patches such as buccal patches.

The composition may be a tablet composition or a capsule composition. Tablet compositions can contain a unit dosage of active compound together with an inert diluent or carrier such as a sugar or sugar alcohol, eg; lactose, sucrose, sorbitol or mannitol; and/or a non-sugar derived diluent such as sodium carbonate, calcium phosphate, calcium carbonate, or a cellulose or derivative thereof such as microcrystalline cellulose (MCC), methyl cellulose, ethyl cellulose, hydroxypropyl methyl cellulose, and starches such as corn starch. Tablets may also contain such standard ingredients as binding and granulating agents such as polyvinylpyrrolidone, disintegrants (e.g., swellable crosslinked polymers such as crosslinked carboxymethylcellulose), lubricating agents (e.g., stearates), preservatives (e.g., parabens), antioxidants (e.g., BHT), buffering agents (for example phosphate or citrate buffers), and effervescent agents such as citrate/bicarbonate mixtures. Such excipients are well known and do not need to be discussed in detail here.

Tablets may be designed to release the drug either upon contact with stomach fluids (immediate release tablets) or to release in a controlled manner (controlled release tablets) over a prolonged period of time or with a specific region of the GI tract.

The pharmaceutical compositions typically comprise from approximately 1% (w/w) to approximately 95%, preferably % (w/w) active ingredient and from 99% (w/w) to 5% (w/w) of a pharmaceutically acceptable excipient (for example as defined above) or combination of such excipients. Preferably, the compositions comprise from approximately 20% (w/w) to approximately 90% (w/w) active ingredient and from 80% (w/w) to 10% of a pharmaceutically excipient or combination of excipients. The pharmaceutical compositions comprise from approximately 1% to approximately 95%, preferably from approximately 20% to approximately 90%, active ingredient. Pharmaceutical compositions according to the invention may be, for example, in unit dose form, such as in the form of ampoules, vials, suppositories, pre-filled syringes, dragées, powders, tablets or capsules.

Tablets and capsules may contain, for example, 0-20% disintegrants, 0-5% lubricants, 0-5% flow aids and/or 0-99% (w/w) fillers/or bulking agents (depending on drug dose). They may also contain 0-10% (w/w) polymer binders, 0-5% (w/w) antioxidants, 0-5% (w/w) pigments. Slow release tablets would in addition typically contain 0-99% (w/w) release-controlling (e.g. delaying) polymers (depending on dose). The film coats of the tablet or capsule typically contain 0-10% (w/w) polymers, 0-3% (w/w) pigments, and/or 0-2% (w/w) plasticizers.

The composition may be a parenteral composition. Parenteral formulations typically contain 0-20% (w/w) buffers, 0-50% (w/w) cosolvents, and/or 0-99% (w/w) Water for Injection (WFI) (depending on dose and if freeze dried). Formulations for intramuscular depots may also contain 0-99% (w/w) oils.

The pharmaceutical formulations may be presented to a patient in “patient packs” containing an entire course of treatment in a single package, usually a blister pack.

The compounds of the Formula (1) will generally be presented in unit dosage form and, as such, will typically contain sufficient compound to provide a desired level of biological activity. For example, a formulation may contain from 1 nanogram to 2 grams of active ingredient, e.g., from 1 nanogram to 2 milligrams of active ingredient. Within these ranges, particular sub-ranges of compound are 0.1 milligrams to 2 grams of active ingredient (more usually from 10 milligrams to 1 gram, e.g., 50 milligrams to 500 milligrams), or 1 microgram to 20 milligrams (for example 1 microgram to 10 milligrams, e.g., 0.1 milligrams to 2 milligrams of active ingredient).

For oral compositions, a unit dosage form may contain from 1 milligram to 2 grams, more typically 10 milligrams to 1 gram, for example 50 milligrams to 1 gram, e.g., 100 milligrams to 1 gram, of active compound.

The active compound will be administered to a patient in need thereof (for example a human or animal patient) in an amount sufficient to achieve the desired therapeutic effect (effective amount). The precise amounts of compound administered may be determined by a supervising physician in accordance with standard procedures.

### EXAMPLES

The invention will now be illustrated, but not limited, by reference to the following examples. Some compounds of Formula (I) and Formula (Ia) and derivatives or synthetic intermediates thereof can be prepared in accordance with synthetic methods known to the skilled person. In some embodiments, the invention provides a process for the preparation of a compound as defined in Formula (I) and Formula (Ia) above. Certain compounds of the invention may be prepared according to the methods described in WO 2021/165346.

### Synthesis of (2R,4S)-4-((6-(3-((2,5-dichloro-3-(hydroxymethyl)phenyl)sulfonamido)-2,6-difluorophenyl)quinazolin-2-yl)amino)-N-(2-methoxyethyl)pyrrolidine-2-carboxamide (Compound 3)

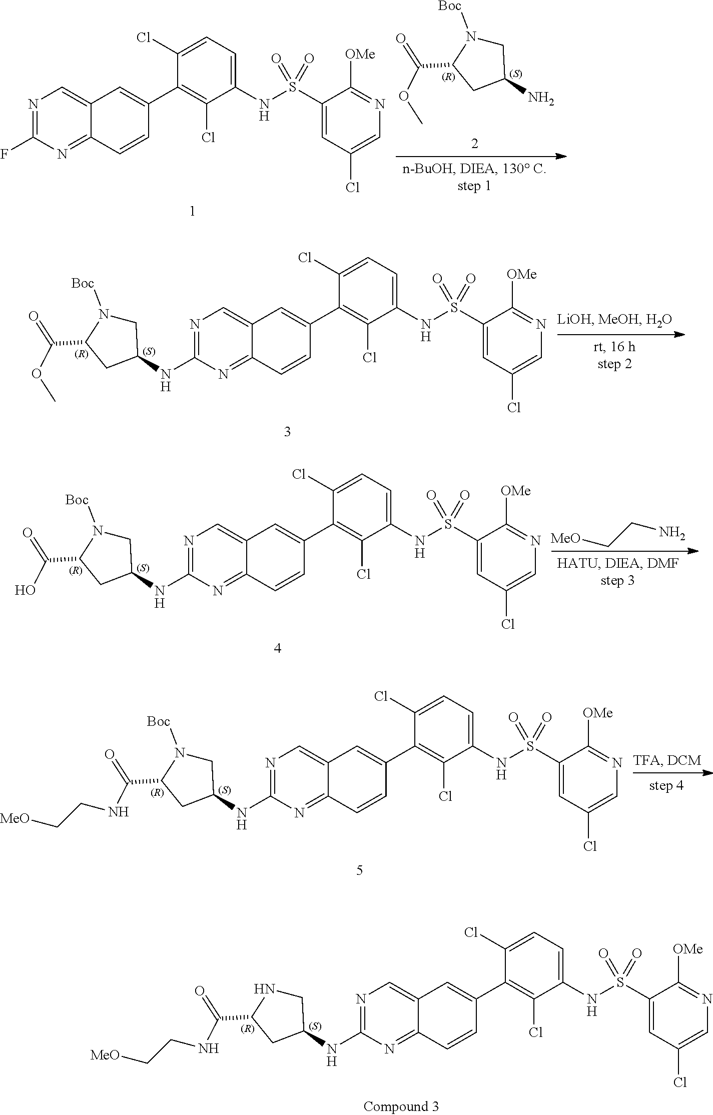

### Synthesis of 5-chloro-N-(2,4-dichloro-3-(2-(((1R,3R)-3-(((2-methoxyethyl)amino)methyl)cyclopentyl)amino)quinazolin-6-yl)phenyl)-2-methoxypyridine-3-sulfonamide (Compound 7)

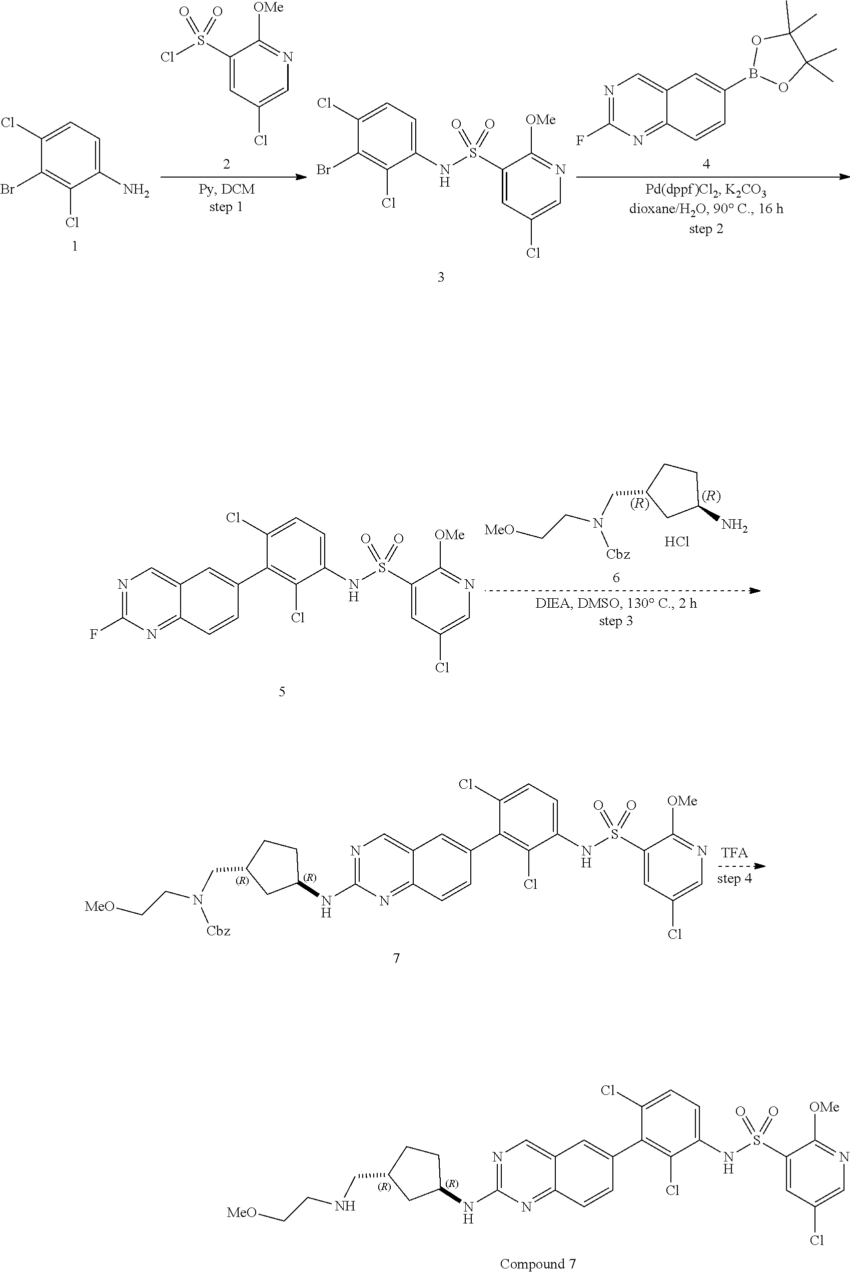

### Synthesis of (2R,4S′)-4-((6-(3-((5-chloro-2-methoxypyridine)-3-sulfonamido)-2,6-difluorophenyl)quinazolin-2-yl)amino)-N-(2-methoxyethyl)pyrrolidine-2-carboxamide (Compound 15)

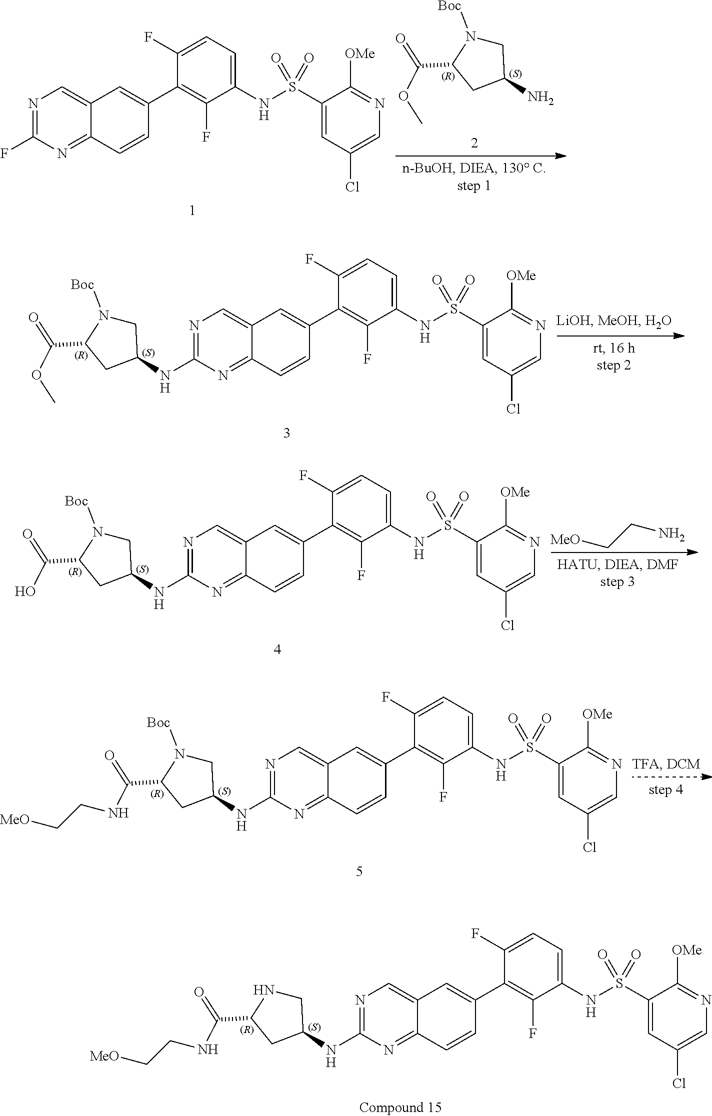

### Synthesis of (1R,3R)-3-((6-(2,6-dichloro-3-((5-chloro-2-methoxypyridine)-3-sulfonamido)phenyl)quinazolin-2-yl)amino)cyclopentane-1-carboxamide (Compound 16)

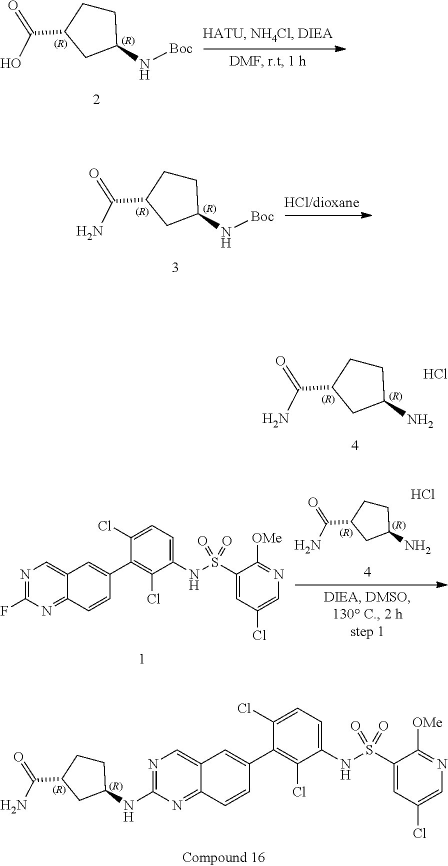

### Synthesis of (R)-3-((6-(2,6-dichloro-3-((5-chloro-2-methoxypyridine)-3-sulfonamido)phenyl)quinazolin-2-yl)amino)-N-(2-methoxyethyl)pyrrolidine-1-carboxamide (Compound 18)

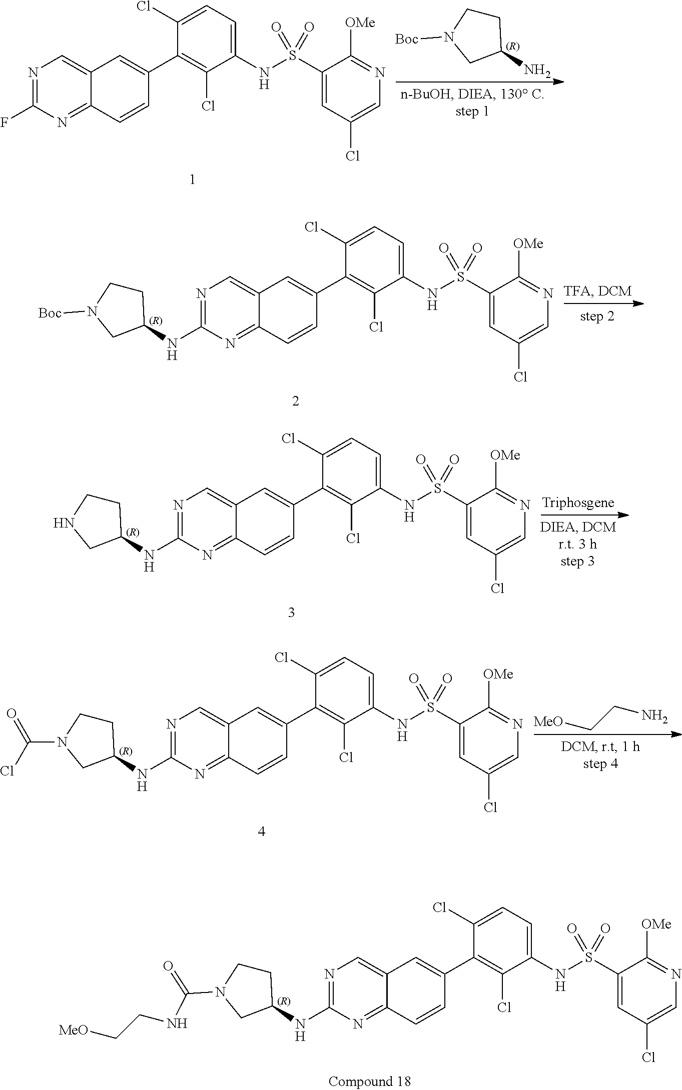

### Synthesis of (1R,4R)-4-((6-(2,6-dichloro-3-((5-chloro-2-methoxypyridine)-3-sulfonamido)phenyl)quinazolin-2-yl)amino)-N-(2-methoxyethyl)cyclohexane-1-carboxamide (Compound 21)

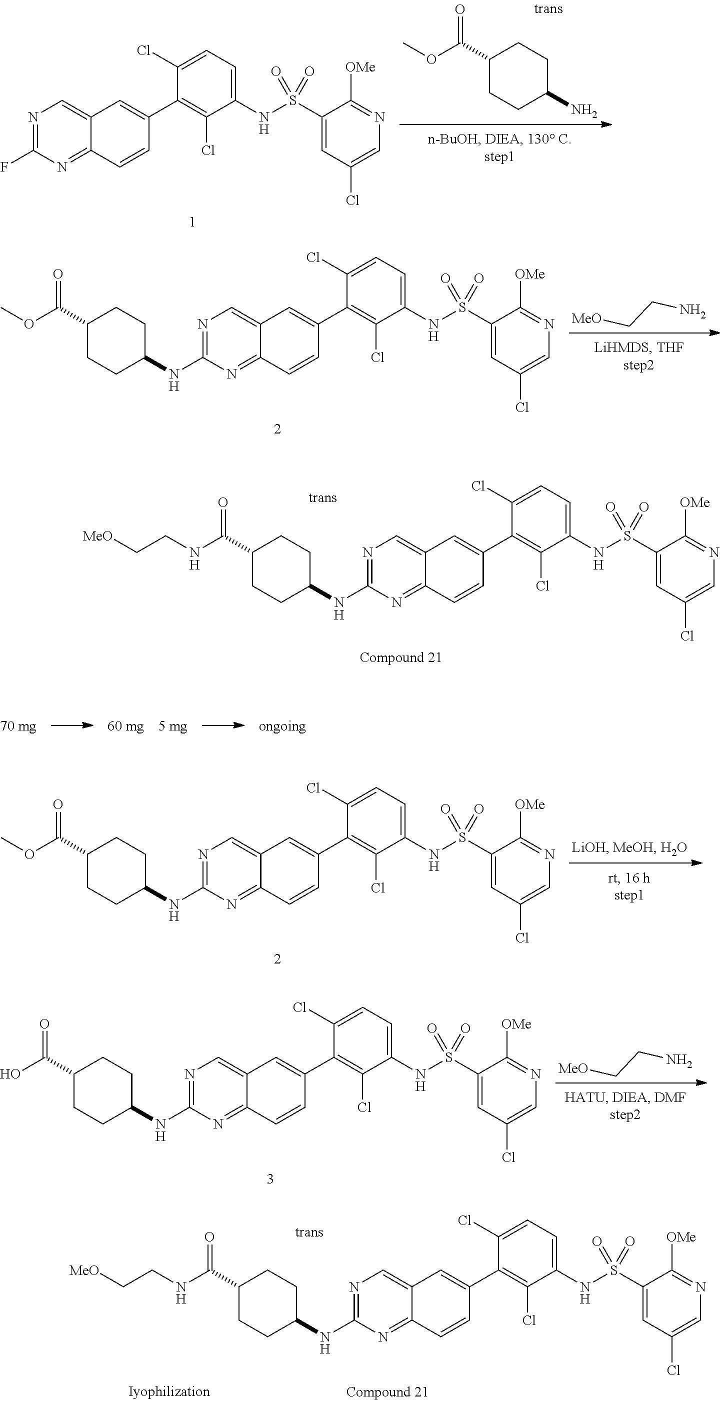

### Synthesis of 5-chloro-N-(2,4-dichloro-3-(2-((1,1-dioxidotetrahydrothiophen-3-yl)amino)quinazolin-6-yl)phenyl)-2-methoxypyridine-3-sulfonamide (Compound 27)

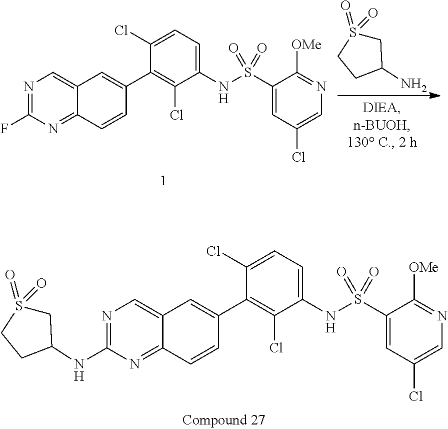

### Synthesis of 5-chloro-N-(2,4-dichloro-3-(2-((2-(dimethylamino)ethyl)amino)quinazolin-6-yl)phenyl)-2-methoxypyridine-3-sulfonamide (Compound 30)

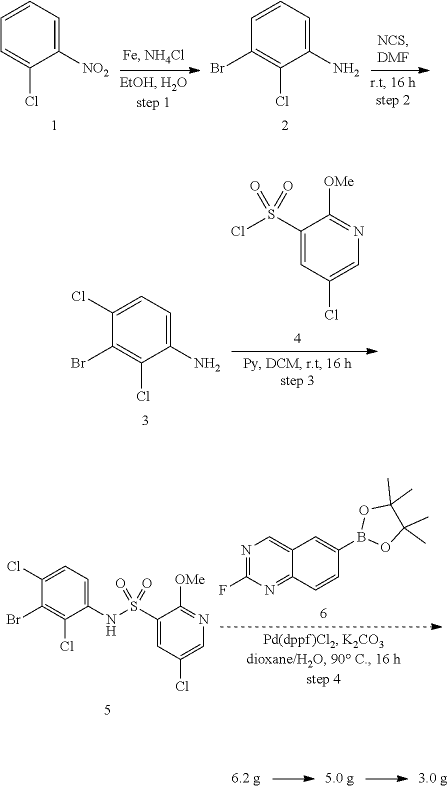

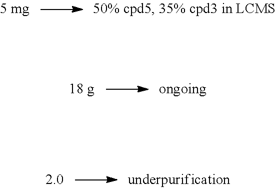

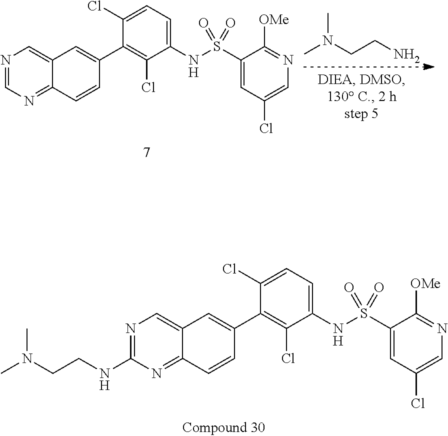

### Synthesis of 2,5-dichloro-N-(3-(2-(((1R,4R)-4-(dimethylamino)cyclohexyl)amino)quinazolin-6-yl)-2,4-difluorophenyl)-3-(hydroxymethyl)benzenesulfonamide (Compound 53)

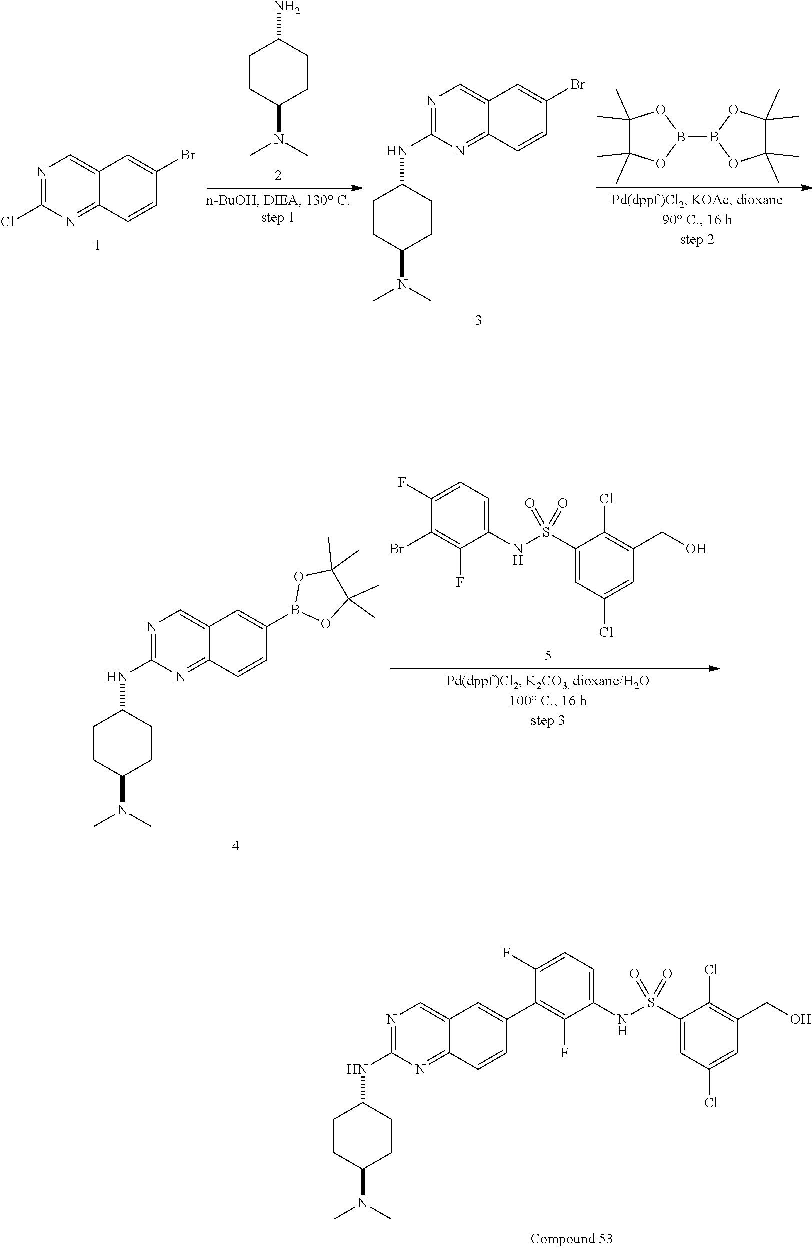

### Synthesis of (1R,3R)-3-((6-(3-((5-chloro-2-methoxypyridine)-3-sulfonamido)-2,6-difluorophenyl)-8-ethylquinazolin-2-yl)amino)-N-(2-methoxyethyl)cyclopentane-1-carboxamide (Compound 54)

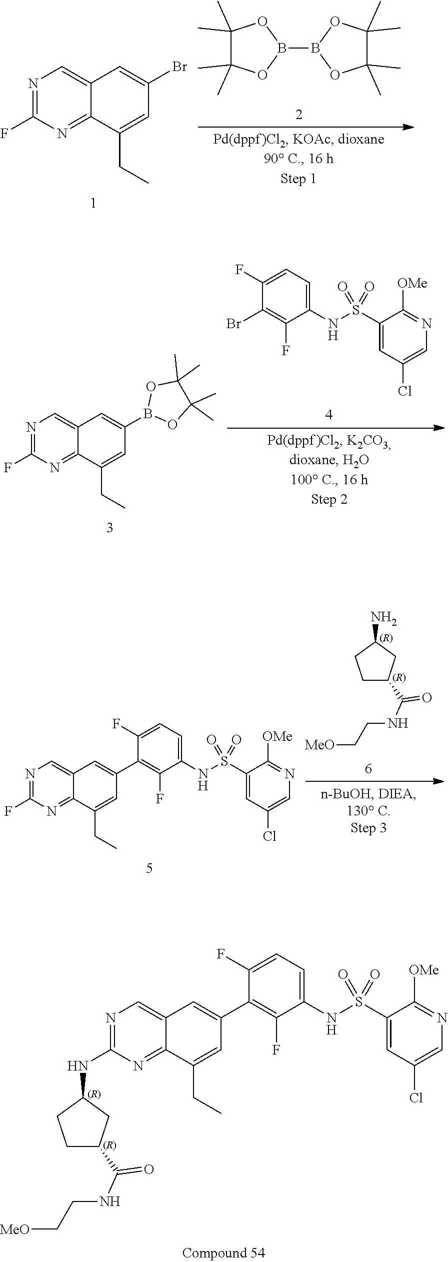

### Synthesis of 2-chloro-N-(3-(2-(((1R,4R)-4-(dimethylamino)cyclohexyl)amino)quinazolin-6-yl)-2,4-difluorophenyl)benzenesulfonamide (Compound 70)

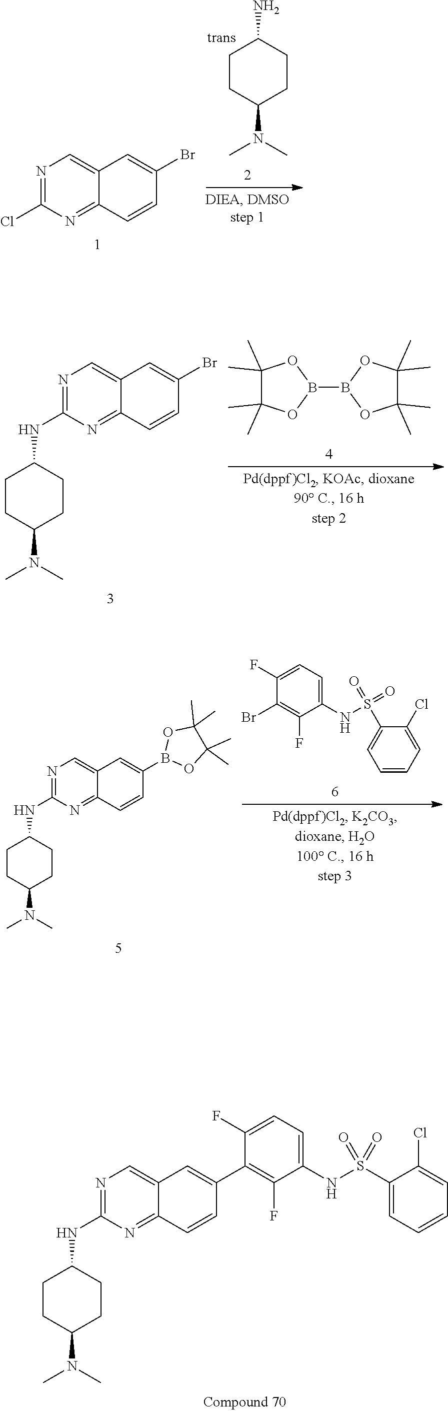

### Synthesis of N-(3-(2-(((1R,4R)-4-aminocyclohexyl)amino)quinazolin-6-yl)-2,4-difluorophenyl)-2-chlorobenzenesulfonamide (Compound 71)

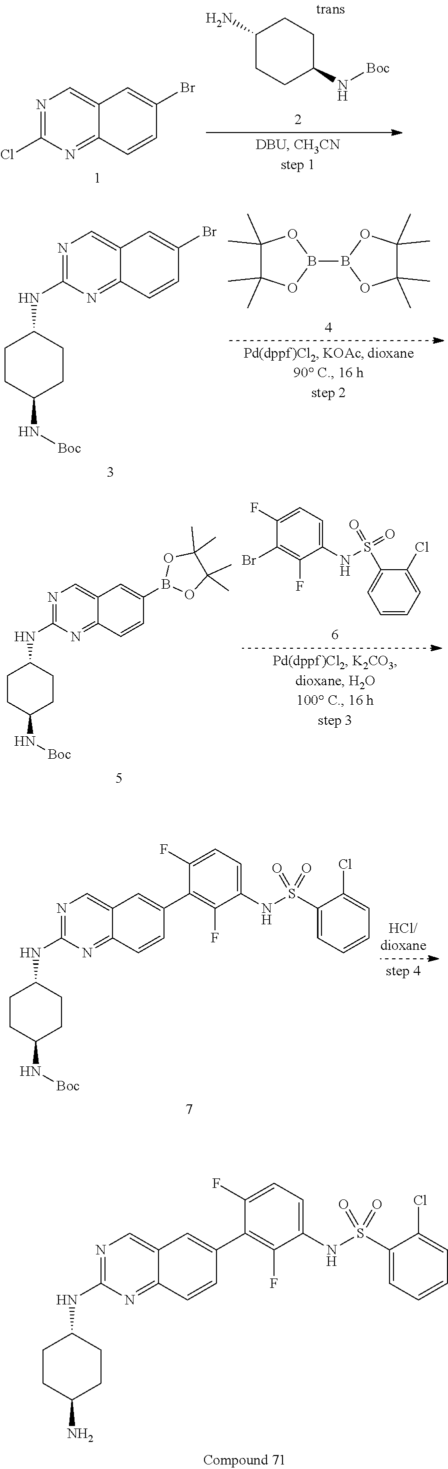

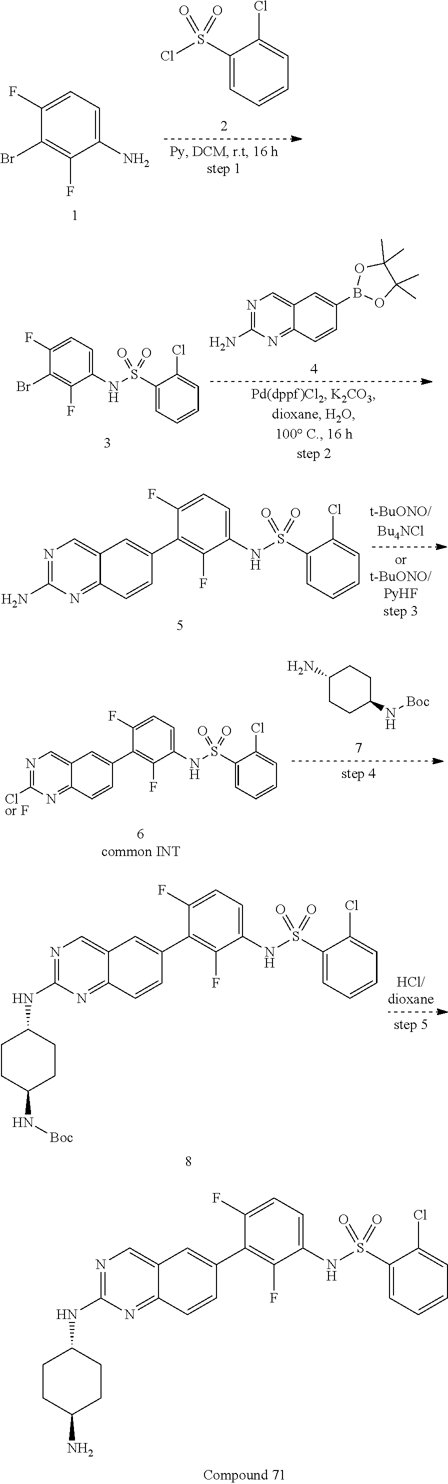

For GCN2 enzyme assays, testing compounds were prepared at proper concentrations in DMSO solution. IC₅₀ values were calculated from a remaining activity using read conversion ratio. IC₅₀ values of exemplary compounds are shown in Table 2.

| Compound Number | GCN2 IC₅₀ (nM) |
|---|---|
| 1 | 10.4 |
| 2 | 2.63 |
| 3 | 5.43 |
| 4 | 3.94 |
| 5 | 11.4 |
| 6 | 3.36 |
| 7 | 12.6 |
| 11 | 2.12 |
| 12 | 7.62 |
| 13 | 3.96 |
| 14 | 3.39 |
| 15 | 4.18 |
| 16 | 11.7 |
| 17 | 9.17 |
| 18 | 41.6 |
| 20 | 10.2 |
| 21 | 31.9 |
| 23 | 6.66 |
| 27 | 25.3 |
| 35 | 8.82 |
| 38 | 7.32 |
| 40 | 8.33 |
| 45 | 88.3 |
| 48 | 3.23 |
| 52 | 5.13 |
| 53 | 1.11 |
| 54 | 34.3 |
| 57 | 3.95 |
| 61 | 2.35 |
| 62 | 1.64 |
| 64 | 26.8 |
| 65 | 43.7 |
| 66 | 63.2 |
| 68 | 39.8 |
| 70 | 81.2 |
| 71 | 123 |
| 73 | 19.4 |
| 75 | 367 |

1. A compound according to Formula (I): 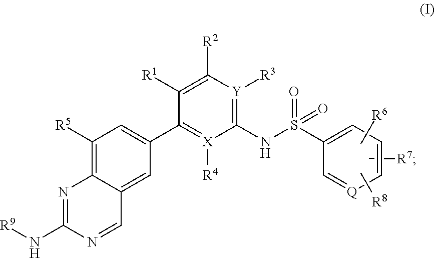
or a stereoisomer, tautomer, or a pharmaceutically acceptable salt thereof, wherein:
R¹, R², R³ and R⁴ are independently selected from the group consisting of H, halo and C₁₋₃ alkyl;
R⁵ is H or C₁₋₃ alkyl;
R⁶, R⁷ and R⁸ are independently selected from the group consisting of H, halo, C₁₋₆ alkyl, and C₁₋₆ alkoxy, wherein the C₁₋₆ alkyl is optionally substituted with OH and the C₁₋₆ alkoxy is optionally substituted with C₁₋₆ alkoxy or NR¹⁰R¹¹;
R⁹ is H, C₁₋₆ alkyl, C₅₋₆ cycloalkyl or 4- to 6-membered heterocyclyl, wherein the C₁₋₆ alkyl is substituted with NR¹⁰R¹¹, and the C₅₋₆ cycloalkyl and 4- to 6-membered heterocyclyl are optionally substituted with one or two substituents each independently selected from the group consisting of oxo, NR¹⁰R¹¹, —C(O)NR¹⁰R¹¹, and C₁₋₆ alkyl optionally substituted with NR¹⁰R¹¹;
R¹⁰ and R¹¹ are independently H or C₁₋₃ alkyl optionally substituted with C₁₋₆ alkoxy; and
X, Y and Q are independently C, CH or N,
provided that when R⁹ is H, and X and Y are C or CH, then at least one of R⁶, R⁷ and R⁸ is C₁₋₆ alkoxy, wherein the C₁₋₆ alkoxy is substituted with C₁₋₆ alkoxy or NR¹⁰R¹¹.

2. The compound of claim 1, or a stereoisomer, tautomer, or a pharmaceutically acceptable salt thereof, wherein R² and R³ are H.

3. The compound of claim 1, or a stereoisomer, tautomer, or a pharmaceutically acceptable salt thereof, wherein 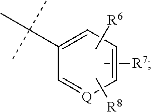
is selected from the group consisting of:

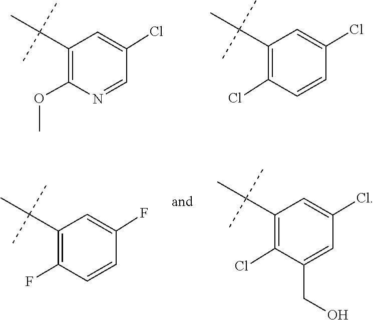

4. The compound of claim 1, or a stereoisomer, tautomer, or a pharmaceutically acceptable salt thereof, wherein R⁹ is H.

5. The compound of claim 1, or a stereoisomer, tautomer, or a pharmaceutically acceptable salt thereof, wherein R⁹ is C₁₋₆ alkyl substituted with NR¹⁰R¹¹.

6. The compound of claim 1, or a stereoisomer, tautomer, or a pharmaceutically acceptable salt thereof, wherein R⁹ is C₅₋₆ cycloalkyl or 4- to 6-membered heterocyclyl, wherein C₅₋₆ cycloalkyl and 4- to 6-membered heterocyclyl are optionally substituted with one or two substituents each independently selected from the group consisting of oxo, NR¹⁰R¹¹, —C(O)NR¹⁰R¹¹, and C₁₋₆ alkyl optionally substituted with NR¹⁰R¹¹.

7. The compound of claim 1, or a stereoisomer, tautomer, or a pharmaceutically acceptable salt thereof, wherein R⁹ is

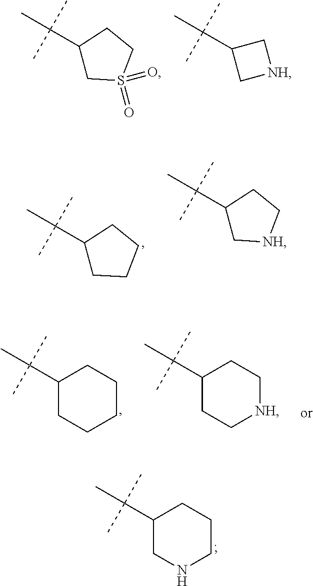

8. The compound of claim 1, or a stereoisomer, tautomer, or a pharmaceutically acceptable salt thereof, wherein R⁹ is 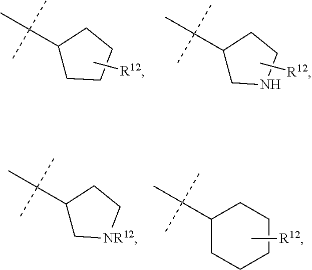
wherein R¹² is selected from the group consisting NR¹⁰R¹¹, —C(O)NR¹⁰R¹¹, or C₁₋₆ alkyl optionally substituted with NR¹⁰R¹¹.

9. The compound of claim 1, or a stereoisomer, tautomer, or a pharmaceutically acceptable salt thereof, wherein X is N.

10. The compound of claim 1, or a stereoisomer, tautomer, or a pharmaceutically acceptable salt thereof, wherein Y is N.

11. A compound according to Formula (Ia): 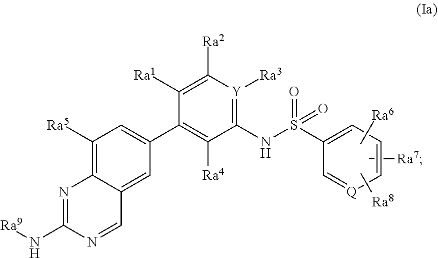
or a stereoisomer, tautomer, or a pharmaceutically acceptable salt thereof, wherein:
Ra¹ and Ra⁴ are halo;
Ra² and Ra³ are independently selected from the group consisting of H, halo and C₁₋₃ alkyl;
Ra⁵ is H or C₁₋₃ alkyl;
Ra⁶, Ra⁷ and Ra⁸ are independently selected from the group consisting of H, halo, C₁₋₆ alkyl, and C₁₋₆ alkoxy, wherein the C₁₋₆ alkyl and C₁₋₆ alkoxy are optionally substituted with OH;
Ra⁹ is H, C₅₋₆ cycloalkyl or 5 or 6-membered heterocyclyl, wherein the C₅₋₆ cycloalkyl and 5 or 6-membered heterocyclyl are optionally substituted with OH; and
Q is C, CH or N.

12. The compound of claim 11, or a stereoisomer, tautomer, or a pharmaceutically acceptable salt thereof, wherein Ra² and Ra³ are H.

13. The compound of claim 11, or a stereoisomer, tautomer, or a pharmaceutically acceptable salt thereof, wherein 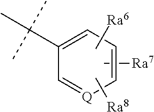
is selected from the group consisting of:

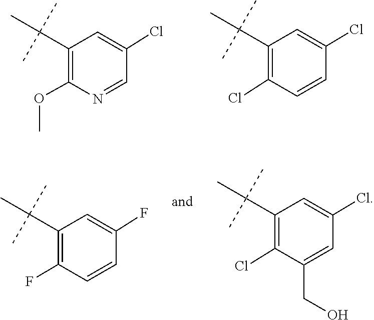

14. The compound of claim 11, or a stereoisomer, tautomer, or a pharmaceutically acceptable salt thereof, wherein Ra⁹ is H.

15. The compound of claim 11, or a stereoisomer, tautomer, or a pharmaceutically acceptable salt thereof, wherein Ra⁹ is C₅₋₆ cycloalkyl optionally substituted with OH.

16. The compound of claim 11, or a stereoisomer, tautomer, or a pharmaceutically acceptable salt thereof, wherein Ra⁹ is 5 or 6-membered heterocyclyl.

17. The compound of claim 11, or a stereoisomer, tautomer, or a pharmaceutically acceptable salt thereof, wherein Ra⁹ is

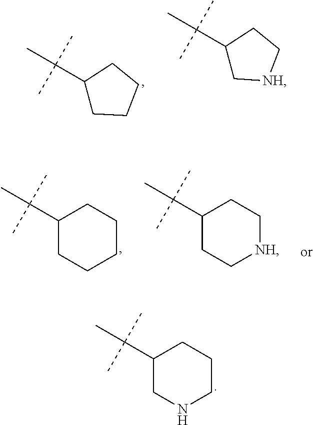

18. A compound selected from the compounds in Table 1 and Table 1a, or a stereoisomer, tautomer, or a pharmaceutically acceptable salt thereof.

19. A pharmaceutical composition comprising a compound of claim 1, or a stereoisomer, tautomer, or a pharmaceutically acceptable salt thereof and a pharmaceutically acceptable excipient.

20. A method of treating a disease or disorder characterised by activation of GCN2 in an individual in need thereof, comprising administering to the individual a therapeutically effective amount of a compound of claim 1, or a stereoisomer, tautomer, or a pharmaceutically acceptable salt thereof.

21-25. (canceled)
